(19) United States
(12) Patent Application Publication
(10) Pub. No.: US 2023/0040919 A1
(43) Pub. Date: Feb. 9, 2023

(54) CROSSLINKED HA-COLLAGEN HYDROGELS AS DERMAL FILLERS

(71) Applicant: ALLERGAN, INC., Irvine, CA (US)

(72) Inventors: Christopher A. TRAINA, Tustin, CA (US); Artem B. KUTIKOV, Costa Mesa, CA (US); Xiaojie YU, Orange, CA (US); Christopher K. HEE, Lake Forest, CA (US); Darin J. MESSINA, Laguna Niguel, CA (US); Julie MORELL, Annecy (FR); Florent DURIEUX, Alex (FR); Olivier GUETTA, Annecy (FR); Jean-Xavier ROCA MARTINEZ, Annecy (FR)

(21) Appl. No.: 17/789,162

(22) PCT Filed: Dec. 28, 2019
(86) PCT No.: PCT/US2020/067230

§ 371 Date: Jun. 24, 2022

Related U.S. Application Data
(60) Provisional application No. 62/953,910, filed on Dec. 26, 2019.

(51) Int. Cl. A61K 8/04; A61K 8/65; A61K 8/73; A61Q 19/08; A61K 8/44; A61K 8/41

(52) U.S. Cl. CPC A61K 8/042; A61K 8/65; A61K 8/735; A61Q 19/08; A61K 8/44; A61K 8/41

(57) ABSTRACT

The present disclosure relates to a crosslinked macromolecular matrix comprising lysine; hyaluronic acid; and collagen; wherein the hyaluronic acid is crosslinked to the collagen by at least one endogenous amine group on the collagen and/or by at least one amine group present on the lysine.

### CROSS-REFERENCE TO RELATED APPLICATIONS

This application claims the benefit of priority of U.S. Patent Application No. 62/953,910, filed Dec. 26, 2019, which is incorporated by reference herein in its entirety.

### FIELD

The present disclosure relates to a crosslinked macromolecular matrix that comprises hyaluronic acid, collagen, and lysine. Such compositions may be used as a tissue filler with enhanced tissue integration.

### BACKGROUND

Aging is a natural process occurring over time which can be affected by genetics and lifestyle factors (recreational drugs, alcohol abuse, tobacco, UVA/UVB exposure, diet). Characteristics of facial skin aging include muscle and fat atrophy, skin laxity, age spots, sagging, and fattening, for example. Slackening of the subcutaneous tissues leads to an excess of skin and ptosis which may lead to the appearance of drooping cheeks and eye lids. Fattening refers to an increase in excess weight by swelling of the bottom of the face and neck. These changes may be associated with dryness, loss of elasticity, and rough texture.

Dermal fillers have been used to improve the appearance of aging skin. Various types of dermal fillers have been developed and used in the treatment or the improvement/correction of imperfections on the body, for example, wrinkles and volume loss due to the effects of aging. Initially, dermal filler compositions comprising bovine collagen, entered the market in the 1970s. Human-derived collagen was approved by the FDA in 2003, which was advantageous over the bovine-derived collagen, which had the potential for allergic reactions in patients. However, the human derived collagen compositions degraded rapidly within 3 to 6 months due to the enzymes within the skin tissues. Thus, patients using these earlier compositions required frequent procedures to maintain the corrective aesthetic appearance they desired.

Hyaluronan, or hyaluronic acid (HA) based fillers were introduced in the 1990's as an alternative to the collagen based dermal fillers. HA is a naturally occurring, water soluble polysaccharide, specifically a glycosaminoglycan, which is a major component of the extra-cellular matrix and is widely distributed in animal tissues. HA has excellent biocompatibility and does not cause allergic reactions when implanted into a patient. In addition, HA has the ability to bind to large amounts of water, making it an excellent volumizer of soft tissues. HA is similar to collagen as, it may also be degraded by endogenous enzymes in the skin. For example, un-crosslinked HA does not have the sufficient duration or physical properties to act as a wrinkle filler, thus crosslinked HA has been used to maximize their longevity in the dermal tissues. As such there is a need for improved dermal fillers.

### SUMMARY

The embodiments herein encompass methods and compositions (e.g., hydrogels or dermal fillers) comprising a crosslinked macromolecular matrix that comprise hyaluronic acid, collagen, and lysine, wherein the hyaluronic acid is crosslinked to the collagen by at least one endogenous amine group on the collagen and/or by at least one amine group present on the lysine.

In some embodiments of any one of each or any of the above- or below-mentioned embodiments, the crosslinked macromolecular matrix further comprises lidocaine. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments, the crosslinked macromolecular matrix further comprises un-crosslinked HA. In some embodiments of any one of each or any of the above- or below-mentioned embodiments, the un-crosslinked HA comprises a concentration of up to about 5% (w/w) within the matrix. In some embodiments of any one of each or any of the above- or below-mentioned embodiments, the un-crosslinked HA comprises a concentration of 0% (w/w), about 1% (w/w), about 2% (w/w), about 3% (w/w), about 4% (w/w), or about 5% (w/w) in the matrix, or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of about 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the macromolecular matrix.

In some embodiments of any one of each or any of the above- or below-mentioned embodiments, the crosslinked macromolecular matrix is stable for at least about 6 months, about 12 months, about 18 months, about 24 months, about 30 months, or about 36 months, or any amount of time in between a range defined by any two aforementioned values. In some embodiments of any one of each or any of the above- or below-mentioned embodiments, the crosslinked macromolecular matrix is stable at a temperature between about 4° C. and about 25° C. In some embodiments of any one of each or any of the above- or below-mentioned embodiments, the crosslinked macromolecular matrix is stable at about 4° C. In some embodiments of any one of each or any of the above- or below-mentioned embodiments, the crosslinked macromolecular matrix is stable at about 25° C. In some embodiments of any one of each or any of the above- or below-mentioned embodiments, the crosslinked macromolecular matrix is stable for about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36 months, or any time in between a range defined by any two aforementioned values.

In some embodiments of any one of each or any of the above- or below-mentioned embodiments, the crosslinked macromolecular matrix has minimal degradation at about 6 months, about 12 months, about 18 months, about 24 months, about 30 months, or about 36 months, or any amount of time in between a range defined by any two aforementioned values.

In some embodiments of any one of each or any of the above- or below-mentioned embodiments, the crosslinked macromolecular matrix comprises an elastic modulus (G′) of about 30 Pa to about 10,000 Pa. In some embodiments of any one of each or any of the above- or below-mentioned embodiments, the matrix comprises an elastic modulus (G′) of about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, about 3000 Pa, about 3100 Pa, about 3200 Pa, about 3300 Pa, about 3400 Pa, about 3500 Pa, about 3600 Pa, about 3700 Pa, about 3800 Pa, about 3900 Pa, about 4000 Pa, about 4100 Pa, about 4200 Pa, about 4300 Pa, about 4400 Pa, about 4500 Pa, about 4600 Pa, about 4700 Pa, about 4800 Pa, about 4900 Pa, about 5000 Pa, about 5100 Pa, about 5200 Pa, about 5300 Pa, about 5400 Pa, about 5500 Pa, about 5600 Pa, about 5700 Pa, about 5800 Pa, about 5900 Pa, about 6000 Pa, about 6100 Pa, about 6200 Pa, about 6300 Pa, about 6400 Pa, about 6500 Pa, about 6600 Pa, about 6700 Pa, about 6800 Pa, about 6900 Pa, about 7000 Pa, about 7100 Pa, about 7200 Pa, about 7300 Pa, about 7400 Pa, about 7500 Pa, about 7600 Pa, about 7700 Pa, about 7800 Pa, about 7900 Pa, about 8000 Pa, about 8100 Pa, about 8200 Pa, about 8300 Pa, about 8400 Pa, about 8500 Pa, about 8600 Pa, about 8700 Pa, about 8800 Pa, about 8900 Pa, about 9000 Pa, about 9100 Pa, about 9200 Pa, about 9300 Pa, about 9400 Pa, about 9500 Pa, about 9600 Pa, about 9700 Pa, about 9800 Pa, about 9900 Pa, or about 10,000 Pa or any elastic modulus in between a range defined by any two aforementioned values.

In some embodiments of any one of each or any of the above- or below-mentioned embodiments, the crosslinked macromolecular matrix comprises a compression force value of about 10 gmf, about 20 gmf, about 30 gmf, about 40 gmf, about 50 gmf, about 60 gmf, about 70 gmf, about 80 gmf, about 90 gmf, about 100 gmf, about 110 gmf, about 120 gmf, about 130 gmf, about 140 gmf, about 150 gmf, about 160 gmf, about 170 gmf, about 180 gmf, about 190 gmf, about 200 gmf, about 210 gmf, about 220 gmf, about 230 gmf, about 240 gmf, about 250 gmf, about 260 gmf, about 270 gmf, about 280 gmf, about 290 gmf, about 300 gmf, about 310 gmf, about 320 gmf, about 330 gmf, about 340 gmf, about 350 gmf, about 360 gmf, about 370 gmf, about 380 gmf, about 390 gmf, about 400 gmf, about 410 gmf, about 420 gmf, about 430 gmf, about 440 gmf, about 450 gmf, about 460 gmf, about 470 gmf, about 480 gmf, about 490 gmf, about 500 gmf, about 510 gmf, about 520 gmf, about 530 gmf, about 540 gmf, about 550 gmf, about 560 gmf, about 570 gmf, about 580 gmf, about 590 gmf or about 600 gmf or any compression force value in between a range defined by any two aforementioned values. In some embodiments of any one of each or any of the above- or below-mentioned embodiments, the crosslinked macromolecular matrix comprises a compression force value of about 100 gmf, about 200 gmf, about 300 gmf, about 400 gmf, about 500 gmf or about 600 gmf or any compression force value in between a range defined by any two aforementioned values.

In some embodiments of any one of each or any of the above- or below-mentioned embodiments, the hyaluronic acid is at a concentration of about 5 mg/ml, about 6 mg/ml, about 8 mg/ml, about 10 mg/ml, about 12 mg/ml, about 14 mg/ml, about 16 mg/ml, about 18 mg/ml, about 20 mg/ml, about 22 mg/ml, about 24 mg/ml, about 26 mg/ml, about 28 mg/ml, about 30 mg/ml, about 32 mg/ml, about 34 mg/ml or about 36 mg/ml or any concentration in between a range defined by any two aforementioned values.

In some embodiments of any one of each or any of the above- or below-mentioned embodiments, the collagen comprises Type I collagen. In some embodiments of any one of each or any of the above- or below-mentioned embodiments, the collagen comprises Type II collagen. In some embodiments of any one of each or any of the above- or below-mentioned embodiments, the collagen comprises Type III collagen. In some embodiments of any one of each or any of the above- or below-mentioned embodiments, the collagen comprises about 1-3% Type I or III collagen. In some embodiments of any one of each or any of the above- or below-mentioned embodiments, the collagen comprises about 0% to about 3% Type II collagen. In some embodiments of any one of each or any of the above- or below-mentioned embodiments, the collagen comprises about 97% to about 99% Type I collagen. In some embodiments of any one of each or any of the above- or below-mentioned embodiments, the collagen comprises a mixture of both Type I and Type III collagen. In some embodiments of any one of each or any of the above- or below-mentioned embodiments, the matrix comprises about 0% to about 3% type III collagen.

In some embodiments of any one of each or any of the above- or below-mentioned embodiments, the crosslinked macromolecular matrix is formulated for injection or use with a needle and/or cannula.

In some embodiments of any one of each or any of the above- or below-mentioned embodiments, the collagen comprises a concentration of about 1 mg/ml, about 2 mg/ml, about 4 mg/ml, about 6 mg/ml, about 8 mg/ml, about 10 mg/ml, about 12 mg/ml, about 14 mg/ml or any concentration in between a range defined by any two aforementioned values. In some embodiments of any one of each or any of the above- or below- mentioned embodiments, the collagen comprises a concentration of about 3 mg/ml. In some embodiments of any one of each or any of the above- or below- mentioned embodiments, the collagen comprises a concentration of about 6 mg/ml. In some embodiments of any one of each or any of the above- or below- mentioned embodiments, the collagen comprises a concentration of about 10 mg/ml. In some embodiments of any one of each or any of the above- or below-mentioned embodiments, the collagen comprises a concentration of about 12 mg/ml.

In some embodiments of any one of each or any of the above- or below-mentioned embodiments, the crosslinked macromolecular matrix further comprises a salt. In some embodiments of any one of each or any of the above- or below-mentioned embodiments, the crosslinked macromolecular matrix comprises NaCl in a range between about 50 mM to about 400 mM. In some embodiments of any one of each or any of the above- or below-mentioned embodiments, the crosslinked macromolecular matrix comprises NaCl, wherein the NaCl comprises a concentration of about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, or about 400 mM, or any concentration in between a range defined by any two aforementioned values. In some embodiments of any one of each or any of the above- or below-mentioned embodiments, the crosslinked macromolecular matrix comprises about 150 mM NaCl. In certain embodiments, the crosslinked macromolecular matrix is free of salt.

In some embodiments of any one of each or any of the above- or below-mentioned embodiments, the crosslinked macromolecular matrix comprises phosphate buffer of about 0.01M, NaCl of about 137 mM and KCl in a concentration of about 2.7 mM.

In some embodiments of any one of each or any of the above- or below-mentioned embodiments, the hyaluronic acid has an average molecular weight of about 20,000 Daltons to about 10,000,000 Daltons. In some embodiments of any one of each or any of the above- or below-mentioned embodiments, the hyaluronic acid has an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons or about 1,000,000 Daltons, or an average molecular weight in between a range defined by any two aforementioned values. In some embodiments of the composition of any one of each or any of the above- or below-mentioned embodiments, the hyaluronic acid comprises an average molecular weight of about 20,000 Daltons to about 10,000,000 Daltons. In some embodiments of any one of each or any of the above- or below-mentioned embodiments, the hyaluronic acid comprises a mixture of hyaluronic acid components with different molecular weights, wherein the mixture comprises hyaluronic acid with a molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

The disclosure also provides a composition that comprises: hyaluronic acid, collagen, lysine, and a buffer; and wherein the composition is an aqueous hydrogel.

In some embodiments of any one of each or any of the above- or below-mentioned embodiments, the hyaluronic acid is crosslinked to the collagen by at least one endogenous amine group on the collagen and/or by at least one amine group present on the lysine. In some embodiments of any one of each or any of the above- or below-mentioned embodiments, the composition further comprises lidocaine. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the composition. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the composition, or any concentration in between a range defined by any two aforementioned values.

In some embodiments of any one of each or any of the above- or below-mentioned embodiments, the composition further comprises un-crosslinked HA. In some embodiments of any one of each or any of the above- or below-mentioned embodiments, the un-crosslinked HA comprises a concentration of up to about 5% (w/w) within the composition. In some embodiments of any one of each or any of the above- or below-mentioned embodiments, the un-crosslinked HA comprises a concentration of about 0% (w/w), about 1% (w/w), about 2% (w/w), about 3% (w/w), about 4% (w/w), or about 5% (w/w) in the composition, or any concentration in between a range defined by any two aforementioned values. In some embodiments of any one of each or any of the above- or below-mentioned embodiments, the un-crosslinked HA comprises a concentration of about 1% (w/w) of the composition. In some embodiments of any one of each or any of the above- or below-mentioned embodiments, the un-crosslinked HA comprises a concentration of about 2% (w/w) of the composition. In some embodiments of any one of each or any of the above- or below-mentioned embodiments, the un-crosslinked HA comprises a concentration of about 5% (w/w) of the composition. In some embodiments of any one of each or any of the above- or below-mentioned embodiments, the un-crosslinked HA improves the extrudability of the composition. In some embodiments of any one of each or any of the above- or below-mentioned embodiments, the buffer is phosphate buffered saline.

In some embodiments of any one of each or any of the above- or below-mentioned embodiments, the hyaluronic acid of the composition comprises an average molecular weight of about 20,000 Daltons to about 10,000,000 Daltons.

In some embodiments of any one of each or any of the above- or below-mentioned embodiments, the hyaluronic acid comprises a mixture of hyaluronic acid components with different molecular weights, wherein the mixture comprises hyaluronic acid with a molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

In some embodiments of any one of each or any of the above- or below-mentioned embodiments, the collagen of the composition comprises collagen type I. In some embodiments of any one of each or any of the above- or below-mentioned embodiments, the collagen comprises collagen type II. In some embodiments of any one of each or any of the above- or below-mentioned embodiments, the collagen comprises collagen type III.

In some embodiments of any one of each or any of the above- or below-mentioned embodiments, the composition is stable for about 6 months, about 12 months, about 18 months, about 24 months, about 30 months, or about 36 months, or any amount of time in between a range defined by any two aforementioned values. In some embodiments of any one of each or any of the above- or below-mentioned embodiments, the composition is stable at about 4° C. In some embodiments of any one of each or any of the above- or below-mentioned embodiments, the composition is stable at about 25° C. In some embodiments of any one of each or any of the above- or below-mentioned embodiments, the composition has minimal degradation at about 6 months, about 12 months, about 18 months, about 24 months, about 30 months, or about 36 months, or any amount of time in between a range defined by any two aforementioned values.

In some embodiments of any one of each or any of the above- or below-mentioned embodiments, the composition further comprises un-crosslinked HA. In some embodiments of any one of each or any of the above- or below-mentioned embodiments, the un-crosslinked HA comprises a concentration of up to about 5% (w/w) within the composition. In some embodiments of any one of each or any of the above- or below-mentioned embodiments, the un-crosslinked HA improves the extrudability of the composition. In some embodiments of any one of each or any of the above- or below-mentioned embodiments, the composition is stable for about 6 months, about 12 months, about 18 months, about 24 months, about 30 months, or about 36 months, or any amount of time in between a range defined by any two aforementioned values. In some embodiments of any one of each or any of the above- or below-mentioned embodiments, the composition is stable at 4° C. In some embodiments of any one of each or any of the above- or below-mentioned embodiments, the composition is stable at 25° C. In some embodiments of any one of each or any of the above- or below-mentioned embodiments, the composition has minimal degradation at 6 months, 12 months, 18 months, 24 months, 30 months, or 36 months or any amount of time in between a range defined by any two aforementioned values.

In some embodiments of any one of each or any of the above- or below-mentioned embodiments, the composition comprises a viscosity of about 4,000 Pa S, about 4100 Pa S, about 4200 Pa S, about 4300 Pa S, about 4400 Pa S, about 4500 Pa S, about 4600 Pa S, about 4700 Pa S, about 4800 Pa S, about 4900 Pa S, about 5000 Pa S, about 5100 Pa S, about 5200 Pa S, about 5300 Pa S, about 5400 Pa S, about 5500 Pa S, about 5600 Pa S, about 5700 Pa S, about 5800 Pa S, about 5900 Pa S, about 6000 Pa S, about 6100 Pa S, about 6200 Pa S, about 6300 Pa S, about 6400 Pa S, about 6500 Pa S, about 6600 Pa S, about 6700 Pa S, about 6800 Pa S, about 6900 Pa S, about 7000 Pa S, about 7100 Pa S, about 7200 Pa S, about 7300 Pa S, about 7400 Pa S, about 7500 Pa S, about 7600 Pa S, about 7700 Pa S, about 7800 Pa S, about 7900 Pa S, about 8000 Pa S, about 8100 Pa S, about 8200 Pa S, about 8300 Pa S, about 8400 Pa S, about 8500 Pa S, about 8600 Pa S, about 8700 Pa S, about 8800 Pa S, about 8900 Pa S, about 9000 Pa S, about 9100 Pa, about 9200 Pa S, about 9300 Pa S, about 9400 Pa S, about 9500 Pa S, about 9600 Pa S, about 9700 Pa S, about 9800 Pa S, about 9900 Pa S, or about 10,000 Pa S or any viscosity in between a range defined by any two aforementioned values.

In some embodiments of any one of each or any of the above- or below-mentioned embodiments, the composition comprises a tan delta parameter (G″/G′) of about 0.01 to about 0.5. In some embodiments of any one of each or any of the above- or below-mentioned embodiments, the composition comprises a tan delta parameter (G″/G′) of about 0.01, about 0.05, about 0.10, about 0.15, about 0.20, about 0.25, about 0.30, about 0.35, about 0.40, about 0.45 or about 0.50 or any tan delta parameter in between a range defined by any two aforementioned values. In some embodiments of the composition of any one of each or any of the above- or below-mentioned embodiments, the buffer comprises phosphate buffered saline.

The disclosure also provides a method of crosslinking hyaluronic acid and collagen. The method comprises dissolving collagen, hyaluronic acid and lysine in an aqueous solution to form an aqueous pre-reaction solution, wherein the aqueous pre-reaction solution comprises a pH between 4 and 6, and preparing a second solution comprising: a water soluble carbodiimide; and an N-hydroxysuccinimide or an N-hydroxysulfosuccinimide; and adding the second solution to the aqueous pre-reaction solution to form a crosslinking reaction mixture, and reacting the crosslinking reaction mixture by crosslinking the hyaluronic acid and the collagen with lysine, wherein the hyaluronic acid is crosslinked to the collagen by at least one endogenous amine group on the collagen and/or by at least one amine group present on the lysine, and wherein the HA and collagen undergo minimal degradation and the structure of the HA and collagen remains intact, thereby forming a crosslinked macromolecular matrix. In some embodiments of any one of each or any of the above- or below- mentioned embodiments, the aqueous pre-reaction solution comprises a pH of about 4.0, about 4.5, about 5.0, about 5.5 or about 6, or any pH in between a range defined by any two aforementioned values. In some embodiments of any one of each or any of the above- or below-mentioned embodiments, the method further comprises providing an activating agent comprising a triazole, a fluorinated phenol, a succinimide, or a sulfosuccinimide.

In some embodiments of any one of each or any of the above- or below-mentioned embodiments, the method further comprises adding lidocaine to the crosslinked macromolecular matrix. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w). about 0.35% (w/w), about 0.37% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments, the method further comprises adding uncrosslinked HA to the crosslinked macromolecular matrix. In some embodiments of any one of each or any of the above- or below-mentioned embodiments, the un-crosslinked HA comprises a concentration of up to about 5% w/w within the crosslinked macromolecular matrix. In some embodiments of any one of each or any of the above- or below-mentioned embodiments, the un-crosslinked HA is added to a concentration of about 0% (w/w), about 1% (w/w), about 2% (w/w), about 3% (w/w), about 4% (w/w), or about 5% (w/w) in the matrix, or any concentration in between a range defined by any two aforementioned values. In some embodiments of any one of each or any of the above- or below-mentioned embodiments, the un-crosslinked HA added to a concentration of about 1% (w/w) in the matrix. In some embodiments of any one of each or any of the above- or below-mentioned embodiments, the un-crosslinked HA is added to a concentration of about 3% (w/w) in the matrix. In some embodiments of any one of each or any of the above- or below-mentioned embodiments, the un-crosslinked HA is added to a concentration of about 5% (w/w) in the matrix.

In some embodiments of any one of each or any of the above- or below-mentioned embodiments, the reacting step is performed between about 4° C. and about 35° C. In some embodiments of any one of each or any of the above- or below-mentioned embodiments, the reacting step is performed at about 4° C., about 5° C., about 7° C., about 9° C., about 11° C., about 13° C., about 15° C., about 17° C., about 19° C., about 21° C., about 23° C., about 25° C., about 27° C., about 29° C., about 31° C., about 33° C., about 35° C., or any temperature in between a range defined by any two aforementioned values. In some embodiments of any one of each or any of the above- or below-mentioned embodiments, the reacting step is performed at about 4° C. or about 22° C.

In some embodiments of any one of each or any of the above- or below-mentioned embodiments, the method further comprises purifying the crosslinked macromolecular matrix, wherein the purifying step is performed using dialysis purification. In some embodiments of any one of each or any of the above- or below-mentioned embodiments, dialysis is performed between about 2° C. and about 30° C. In some embodiments of any one of each or any of the above- or below-mentioned embodiments, dialysis is performed at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C., or any temperature in between a range defined by any two aforementioned values. In some embodiments of any one of each or any of the above- or below-mentioned embodiments, the purifying step is performed at between about 2° C. and about 8° C. In some embodiments of any one of each or any of the above- or below-mentioned embodiments, the purifying step is performed at about 2° C., about 4° C., about 6° C., about 8° C., or any temperature in between a range defined by any two aforementioned values.

In some embodiments of any one of each or any of the above- or below-mentioned embodiments, the method is performed below room temperature. In some embodiments of any one of each or any of the above- or below-mentioned embodiments, the method is performed at a temperature of about 2° C., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., about 34° C., or about 36° C. or a temperature in between a range defined by any two aforementioned values.

In some embodiments of any one of each or any of the above- or below-mentioned embodiments, the pH of the crosslinking reaction mixture is between about 4 to about 6.0. In some embodiments of any one of each or any of the above- or below-mentioned embodiments, the pH of the crosslinking reaction mixture is about 4.0, about 4.5, about 5.0, about 5.5 or about 6.0, or any pH in between a range defined by any two aforementioned values.

In some embodiments of any one of each or any of the above- or below-mentioned embodiments, the pre-reaction solution comprises a salt, wherein the salt comprises sodium chloride at a concentration of about 50 mM, about 75 mM, about 100 mM, about 125 mM, about 150 mM, about 175 mM, about 200 mM, about 225 mM, about 250 mM, about 275 mM, about 300 mM, 325 mM, about 350 mM, about 375 mM, or about 400 mM, or any concentration in between a ranged defined by any two aforementioned values, in the crosslinking reaction mixture.

In some embodiments of any one of each or any of the above- or below-mentioned embodiments, the water soluble carbodiimide is 1-ethyl-3-(3-dimethylaminopropyl)carbodiimide at a concentration of about 20 mM to about 200 mM in the crosslinking reaction mixture. In some embodiments of any one of each or any of the above- or below-mentioned embodiments, the water soluble carbodiimide is 1-ethyl-3-(3-dimethylaminopropyl)carbodiimide at a concentration of about 20 mM, about 40 mM, about 60 mM, about 80 mM, about 100 mM, about 120 mM, about 140 mM, about 160 mM, about 180 mM or about 200 mM, or any concentration in between a range defined by any to aforementioned values.

In some embodiments of the method of any one of each or any of the above- or below-mentioned embodiments, the water soluble carbodiimide and hyaluronic acid is at a mole to mole ratio of water soluble carbodiimide: hyaluronic acid repeat unit between about 0.5 to about 2.0. In some embodiments of any one of each or any of the above- or below-mentioned embodiments, the water soluble carbodiimide and hyaluronic acid is at a mole to mole ratio of water soluble carbodiimide: hyaluronic acid repeat unit of about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0.

In some embodiments of any one of each or any of the above- or below-mentioned embodiments, the lysine and hyaluronic acid are at a mole:mole (lysine:HA repeat unit) ratio between about 0.01 and about 0.6. In some embodiments of any one of each or any of the above- or below-mentioned embodiments, the lysine and hyaluronic acid are at a mole:mole (lysine:HA repeat unit) ratio of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59 or about 0.6.

In some embodiments of any one of each or any of the above- or below-mentioned embodiments, the method further comprises sterilizing the crosslinked macromolecular matrix, the method comprises: transferring the crosslinked macromolecular matrix into a container, for steam sterilization; and sterilizing the hydrogel by steam sterilization. In some embodiments of any one of each or any of the above- or below-mentioned embodiments, the container is a syringe.

In some embodiments of any one of each or any of the above- or below-mentioned embodiments, the method further comprises dialyzing the crosslinked macromolecular matrix, wherein the dialysis is through a membrane having a molecular weight cutoff of about 1000 Daltons to about 100,000 Daltons, and wherein the dialyzing is performed prior to sterilization. In some embodiments of any one of each or any of the above- or below-mentioned embodiments, the dialysis is performed in phosphate buffered saline.

In some embodiments of any one of each or any of the above- or below-mentioned embodiments, the hyaluronic acid in the pre-reaction solution hydrates for at least 60 minutes prior to adding the second solution.

In some embodiments of any one of each or any of the above- or below-mentioned embodiments, the crosslinking reaction mixture is performed for about 16 hours to about 24 hours. In some embodiments of any one of each or any of the above- or below-mentioned embodiments, the crosslinking reaction mixture is performed for about 16 hours, about 18 hours, about 20 hours, about 22 hours, or about 24 hours, or any amount of time within a range defined by any two aforementioned values.

In some embodiments of any one of each or any of the above- or below-mentioned embodiments, the crosslinking reaction is performed at about 2° C. to about 35° C. In some embodiments of any one of each or any of the above- or below-mentioned embodiments, the crosslinking reaction is performed at about 2° C., about 3° C., about 4° C., about 5° C., about 7° C., about 9° C., about 11° C., about 13° C., about 15° C., about 17° C., about 19° C., about 21° C., about 23° C., about 25° C., about 27° C., about 29° C., about 31° C., about 33° C., about 35° C., or any temperature within a range defined by any two aforementioned values.

In some embodiments of any one of each or any of the above- or below-mentioned embodiments, the crosslinking reaction is performed at about 2° C. to about 8° C. In some embodiments of any one of each or any of the above- or below-mentioned embodiments, the crosslinking reaction is performed at about 2° C., about 4° C., about 6° C., or about 8° C., or any temperature within a range defined by any two aforementioned values.

The disclosure also provides a crosslinked macromolecular matrix prepared by a process of any one of the above- of below-mentioned embodiments.

Additionally, the disclosure provides a method of improving an aesthetic quality of an anatomic feature of a human being. The method comprises: injecting a composition into a tissue of the human being to thereby improve the aesthetic quality of the anatomic feature; wherein the composition comprises a crosslinked macromolecular matrix comprising: hyaluronic acid; lysine; and collagen; wherein the hyaluronic acid is crosslinked to the collagen by at least one endogenous amine group on the collagen and/or by at least one amine group present on the lysine.

The disclosure also provides a method of improving the appearance of an individual. The method comprises injecting a composition into a tissue of the individual at an injection site to thereby improve the aesthetic quality of an anatomic feature, wherein infiltrating cells from the tissue integrate into the composition within the injection site, depositing new collagen within the composition; wherein the composition comprises a crosslinked macromolecular matrix comprising hyaluronic acid; lysine; and collagen; wherein the hyaluronic acid is crosslinked to the collagen by at least one endogenous amine group on the collagen and/or by at least one amine group present on the lysine; and wherein the tissue injected by the composition is shown to have tissue integration and collagen deposition and blood vessel formation. In some embodiments of any one of each or any of the above- or below-mentioned embodiments, the composition is injected into a nasolabial fold. In some embodiments of any one of each or any of the above- or below-mentioned embodiments, the method improves symmetry among facial features. In some embodiments of any one of each or any of the above- or below-mentioned embodiments, the method enhances and restores volume to facial features. In some embodiments of any one of each or any of the above- or below-mentioned embodiments, the method restores volume to cheeks/and or temples. In some embodiments, the method augments, corrects, restores or creates volume in the chin, jaw line, or nasolabial fold. In some embodiments of any one of each or any of the above- or below-mentioned embodiments, the composition is injected into tear troughs of the individual. In some embodiments of any one of each or any of the above- or below-mentioned embodiments, the composition is injected into an area comprising dermal atrophy and/or fat pad atrophy. In some embodiments of any one of each or any of the above- or below-mentioned embodiments, the method provides a natural look, feel and movement in the tissue receiving the injection, wherein the composition leads to increased infiltration of collagen from tissue surrounding the injection site. In some embodiments of any one of each or any of the above- or below-mentioned embodiments, there is an enhanced duration of the composition as a result of tissue integration into the injection site. In some embodiments of any one of each or any of the above- or below-mentioned embodiments, the method improves hydration and elasticity of skin surrounding the injection site.

The disclosure also provides a method of increasing tissue infiltration in a dermal filler implant with deposition of collagen. The method comprises injecting a composition into the tissue of an individual, thereby creating a dermal filler depot comprising the composition, wherein the composition comprises a crosslinked macromolecular matrix comprising: hyaluronic acid; lysine; and collagen; wherein the hyaluronic acid is crosslinked to the collagen by at least one endogenous amine group on the collagen and/or by at least one amine group present on the lysine; and wherein cells from the tissue surrounding the dermal filler depot infiltrates the dermal filler depot comprising the composition, wherein the cells integrate into the composition and deposit new collagen into the composition, thereby creating infiltrated tissue within the composition and wherein blood vessels connect the infiltrated tissue within the composition to a blood supply of the individual's body.

In some embodiments of any one of each or any of the above- or below-mentioned embodiments, the collagen comprises collagen type I and/or collagen type III.

In some embodiments of any one of each or any of the above- or below-mentioned embodiments, the composition comprises about 18 mg/ml hyaluronic acid, about 20 mg/mL hyaluronic acid, about 22 mg/ml hyaluronic acid, about 24 mg/ml hyaluronic acid, about 26 mg/ml hyaluronic acid, about 28 mg/ml hyaluronic acid or about 30 mg/ml hyaluronic acid or any concentration in between a range defined by any two aforementioned values. In some embodiments of any one of each or any of the above- or below-mentioned embodiments, the composition comprises about 13 mg/ml hyaluronic acid.

In some embodiments of any one of each or any of the above- or below-mentioned embodiments of the methods, the composition or macromolecular matrix further comprises lidocaine. In some embodiments of any one of each or any of the above- or below-mentioned embodiments, the lidocaine is at a concentration in between a range of 0.15% (w/w) to 0.45% (w/w) in the matrix. In some embodiments of any one of each or any of the above- or below-mentioned embodiments, the lidocaine is at a concentration in between a range of 0.27% (w/w) to 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w). about 0.35% (w/w), about 0.37% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments of the methods, the composition or macromolecular matrix further comprises un-crosslinked HA. In some embodiments of any one of each or any of the above- or below-mentioned embodiments, the un-crosslinked HA comprises a concentration of up to about 5% (w/w) within the composition or matrix. In some embodiments of any one of each or any of the above- or below-mentioned embodiments of the methods, the un-crosslinked HA comprises a concentration of about 0% (w/w), about 1% (w/w), about 2% (w/w), about 3% (w/w), about 4% (w/w), or about 5% (w/w) in the composition or the matrix, or any concentration in between a range defined by any two aforementioned values. In some embodiments of any one of each or any of the above- or below-mentioned embodiments of the methods, the un-crosslinked HA comprises a concentration of about 1% (w/w) in the composition or the matrix. In some embodiments of any one of each or any of the above- or below-mentioned embodiments of the methods, the un-crosslinked HA comprises a concentration of about 2% (w/w) in the composition or the matrix. In some embodiments of any one of each or any of the above- or below-mentioned embodiments of the methods, the un-crosslinked HA comprises a concentration of about 5% (w/w) in the composition or the matrix.

### BRIEF DESCRIPTION OF THE DRAWINGS

FIG. 1 shows in vitro cell activity cells that were in close contact with HA/collagen crosslinked hydrogel formulations.

FIG. 2 shows an Actin Filament Alignment Index (2A), Cell Length to Width Ratio (2B), and Convex Hull to Cell Area Ratio (2C) of fibroblasts cultured on HA-only hydrogels or HA/Collagen crosslinked hydrogels is depicted. HA-Collagen hydrogels (24:6 HA:Collagen) formulated at a 5° C. hydration temperature (Formulation X) exhibit significantly higher actin filament alignment index, cell length to width ratio, and convex hull to cell area ratio than similar hydrogels formulated at a 22° C. hydration temperature (Formulation VI). *p<0.05 by ANOVA with Tukey post-hoc analysis. A HA-Collagen hydrogel with 20:6 HA:Collagen formulated at a 5° C. hydration temperature exhibits particularly high actin filament alignment index, cell length to width ratio, and convex hull to cell area ratio when compared to HA-only gel (Formulation XIX). *p<0.05 by ANOVA with Tukey post-hoc analysis. 2D shows a ranking of HA-collagen hydrogels as a function of the Euclidian distance (3-dimensional space comprising actin filament alignment index, cell length to width ratio, and convex hull to cell area ratio) from an HA-only hydrogel. Increasing Euclidian distance indicates improved cell spreading and adhesion compared to the low adhesion HA-only gel. Overall, hydrogels formulated at a 5° C. hydration temperature exhibit greater Euclidian distance from the HA-only gel.

FIG. 3 shows a lift profile of Formulation I vs. Formulation II vs. Formulation III (mean+/−SEM).

FIG. 4 shows a lift profile of Formulation XV vs. Formulation III (mean+/−SEM).

FIG. 5 shows a lift profile of Formulation II vs. Formulation XV vs. Formulation XVI (mean+/−SEM).

FIG. 6 shows tissue integration of hydrogels with 4 mg/mL collagen and increasing HA concentrations. (6A) H&E, (6B) Collagen 1a, (6C) Vimentin, (6D) Procollagen 1, (6E) CD31. H&E staining demonstrates reductions in tissue in-growth with increasing HA concentration. As shown, dense Collagen 1a staining is observed in the 13 mg/mL HA formulation (Formulation I). The 20 mg/mL HA formulation shows a reduction in Collagen 1a fill and the 25 mg/mL HA formulation shows large areas devoid of Collagen 1a deposition. Vimentin-positive fibroblast\fibrocyte infiltration was observed in all formulations, with the degree of infiltration decreasing with increasing HA concentration. Procollagen I staining appeared to be reduced in the low HA formulation (Formulation I) compared to the 20 mg/mL and 25 mg/mL HA formulations. The presence of Procollagen I staining in the 20 mg/mL and 25 mg/mL HA formulations may indicate continued collagen deposition over time. Vascularity within the hydrogel boluses was observed in the 20 mg/mL and 25 mg/mL HA formulations, as indicated by positive CD31 staining. CD31 staining was not performed on the 13 mg/mL HA formulation.

FIG. 7 shows tissue integration of hydrogels prepared with a higher proportion of lower molecular weight HA over high molecular weight HA. (A) Colloidal Iron, (B) Collagen 1a, (C) Vimentin, (D) Procollagen 1, (E) CD31. Colloidal iron staining demonstrates tissue integration at the margins of the Formulation XV hydrogel and robust tissue integration throughout the entire Formulation XVI gel bolus. Dense Collagen 1a deposition was observed on the dorsal aspect of the Formulation XV bolus but the deposition did not completely fill the gel. Fine strands of Collagen 1a positive tissue are observed throughout the Formulation XVI hydrogel. Vimentin-positive fibroblast\fibrocyte infiltration was observed in all formulations. The majority of the Formulation XVI bolus was infiltrated with vimentin-positive cells. Procollagen I staining was present in both the Formulation XV and Formulation XVI gels. The presence of Procollagen I staining may indicate continued collagen deposition over time. Vascularity within the hydrogel boluses was observed in both formulations (arrowheads). The Formulation XVI formulation exhibited the most robust vascularization throughout the bolus.

FIG. 8 shows tissue integration of hydrogels containing 24 mg/mL HA and 6 mg/mL collagen prepared at room temperature (Formulation VI) and at 5° C. (Formulation X) hydration temperatures. Collagen 1a staining shows fine collagen distribution around the periphery of Formulation VI hydrogel with limited deposition surrounding the particles of hydrogel. Collagen 1a staining of the Formulation X gel shows robust collagen deposition around the periphery of the hydrogel with dense collagen deposition around the particles of hydrogel.

FIG. 9 shows Hematoxylin and Eosin (H&E) and Immunohistochemical (IHC) Staining of Hydrogel Explants 12 Weeks After Subcutaneous Injection in Rats. H&E staining shows tissue deposition intimately associated with hydrogel particles in Formulation XIX while sparse tissue deposition is observed around large hydrogel deposits in the HA only hydrogel. Vimentin staining more extensive fibrocyte/fibroblasts infiltration into the Formulation XIX hydrogel bolus than the HA only gel. The Formulation XIX bolus is also more highly vascularized than the HA only bolus, as shown by the extensive CD31 positive labeling. The enhanced cell infiltration and vascularization of the Formulation XIX bolus enables more dense and uniform deposition of tissue within the bolus, as shown by the Collagen I labeling.

FIG. 10 shows immunohistochemical (IHC) quantification of positive staining area shows increased levels of vimentin (fibroblasts), collagen I, and CD31 (blood vessels) in the bolus of Formulation XIX hydrogel after 12 weeks subcutaneous implantation in rats, as compared to HA only hydrogel.

FIG. 11 shows lift capacity in a subcutaneous injection model in rats. Formulation XIX exhibits similar lift capacity to a 24 mg/mL HMW HA-only gel from 4 to 12 weeks. As shown, the formulation exhibits enhanced tissue integration while retaining lift capacity similar to HA only gels.

FIG. 12 shows 28 week lift capacity data of crosslinked HA-Collagen gels. The lift capacity of the HA only gel is steadily decreasing over time. The lift capacity of the HA-Collagen gels has remained stable from 12 to 28 weeks. Without limiting the disclosure, this may indicate that the HA-Collagen gels have longer duration of treatment effect than HA only gels. Enhanced duration may be a result of the better integration and tissue ingrowth. As shown, Formulation XIX in particular has significantly better tissue ingrowth than HA only gels.

FIG. 13 shows differences that were observed in 24:6 HA to collagen gels before and after autoclaving. The 24:6 HA to collagen gels (run in duplication as Sample 1 and Sample 2) had overall less cell viability. However, the autoclaved and non-autoclaved formulations all exhibit higher cell viability than the HA only gel. As shown, are experiments done in duplication (Sample 1 and Sample 2) of gels containing 24:6 HA to collagen, before (B) and after (BA) autoclaving. A small but significant difference in cell viability was observed in Sample 1, whereas no significant difference was observed in Sample 2 after autoclaving.

FIG. 14 shows H&E staining of gel filler boluses following 4 weeks subcutaneous implantation in a rat model. Formulation XXII (A; 20 mg HA: 4 mg Collagen, 5° C. hydration) exhibits similar or better tissue integration than Formulation XIX (B; 20 mg HA:6 mg Collagen, 5° C. hydration). Blinded scoring by a pathologist further demonstrates the enhanced integration of Formulation XXII with a score of 2.33 versus a score of 1.83 for Formulation XIX. A higher score indicates better tissue integration. In contrast, Formulation XX (C; 20 mg HA:10 mg Collagen, 25° C. synthesis) exhibits worse tissue integration than Formulation XXII and Formulation XIX. The tissue integration score of Formulation XX is 1.13. This result further demonstrates that tissue integration does not follow a linear trend with collagen concentration. Instead there are optimal synthesis conditions and collagen concentrations that achieve enhanced tissue responses.

FIG. 15 shows in vitro cell viability of human dermal fibroblasts cultured with HA only and HA-Collagen (Formulation XXII and Formulation XXIII) gels.

FIG. 16 shows image analysis of the length to width ratio of human dermal fibroblasts cultured with HA only or HA-Collagen gels (Formulation XXII and Formulation XXIII).

FIG. 17 shows tissue integration scoring of the gel bolus following 4 weeks subcutaneous implantation of Formulation XXII and Formulation XXIII or HA only controls in rats.

FIG. 18 shows Collagen 1a staining of tissue integration of Formulation XXII and Formulation XXIII compared to HA only gel.

FIG. 19 shows quantification of the percent positive area of Collagen 1a staining within the hydrogel bolus after 4 week subcutaneous implantation in rats of Formulation XXII.

FIG. 20 shows confocal micrographs of human dermal fibroblasts cultured on HA only, Formulation XXII, or Formulation XXIII gels for 48 hours. The samples were stained for HA Binding Protein, Hoechst, and wheat germ agglutinin (cell membrane).

FIG. 21 shows immunohistochemistry analysis of the tissue response to HA only and HA-Collagen hydrogels (Formulation XXII and Formulation XXIII) after 4 weeks subcutaneous implantation in rats.

FIG. 22 shows 52 week lift capacity data of Formulation XXII compared to HA only gel.

FIG. 23 shows 26 week lift capacity data of Formulation XXIII compared to HA only gel.

FIG. 24 shows confocal micrographs of human dermal fibroblasts cultured on HA only, Formulation XXVI, or Formulation XXV gels for 48 hours. The samples were stained for HA Binding Protein, Hoechst, and wheat germ agglutinin (cell membrane).

FIG. 25 shows two photon imaging of the second harmonic generation signal (white) and tissue autofluorescence (green) in rats treated with subcutaneous bolus injections of HA only, Formulation XXV, or Formulation XXIII after 12 weeks.

FIG. 26 shows immunohistochemistry analysis of the tissue response to Formulation XXV after 4 weeks subcutaneous implantation in rats.

FIG. 27 shows immunohistochemistry analysis of the tissue response to Formulation XXVI after 4 weeks subcutaneous implantation in rats.

FIG. 28 shows 30 week lift capacity data of Formulation XXV and XXVI compared to HA only gel.

### DETAILED DESCRIPTION

The section headings used herein are for organizational purposes only and are not to be construed as limiting the subject matter described.

Where the definition of terms as used in the specification departs from the commonly used meaning of the term, applicant intends to utilize the definitions provided herein, unless specifically indicated.

Disclosed herein are a crosslinked macromolecular matrix, a composition comprising a crosslinked macromolecular matrix, methods of making the crosslinked macromolecular matrix and methods of improving the appearance of an individual. Fillers comprising the crosslinked macromolecular matrix described in the embodiments, have immediate filling and lifting qualities post-injection which may be followed by tissue integration into the site of injection, which may result in a long-term and natural effect.

Advantageously, the crosslinking method provides HA/collagen materials with adjustable physical properties which give rise to a range of filling and lifting characteristics, thus allowing such materials to be injected into a range of tissue depths, facial areas, and for different purposes (volumizing, severe wrinkles, fine lines, etc.). Furthermore, the synthesis method allows for control of cell infiltration into the injected bolus from the surrounding tissue through covalent incorporation of collagen into the crosslinked hydrogel. Additionally, the crosslinking may also protect the collagen from denaturation. The combination of lifting and tissue integration properties are expected to provide superior facial aesthetic enhancement with a natural feel, appearance, and movement. As described herein, are methods to improve the quality of the fillers that lead to a hybrid material which could outperform previous collagen fillers and current HA fillers.

Unless defined otherwise, all technical and scientific terms used herein have the same meaning as commonly understood by one of ordinary skill in the art to which the inventions pertain.

The terms “a,” “an,” “the” and similar referents used in the context of describing the inventions (especially in the context of the following claims) are to be construed to cover both the singular and the plural, unless otherwise indicated herein or clearly contradicted by context. “About” as used herein when referring to a measurable value is meant to encompass variations of +20% or +10% , more preferably +5% , even more preferably +1% , and still more preferably +0. 1% from the specified value.

As used herein, except where the context requires otherwise, the term ‘comprise’ and variations of the term, such as “comprising,” “comprises” and “comprised,” are not intended to exclude further additives, components, integers or steps.

A “crosslinked macromolecular matrix” refers to the matrix formed by crosslinking HA and collagen. The HA and collagen may be crosslinked by activating native carboxylic acid moieties on the HA and collagen so that such moieties can react with endogenous amine groups present on the collagen. Additionally, lysine may be added as a carboxylic acid/di-amine crosslinker to further enhance the crosslinking of HA and collagen. Such addition of lysine permits the tuning of the physical properties of resulting hydrogels. A crosslinked macromolecular matrix may be used in a composition or formulation for medical aesthetics (e.g., as an aesthetic or dermal filler).

“Hyaluronic acid” or “Hyaluronan” as described herein refers to a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the extracellular matrix, to act as a filler material; and to participate in tissue repair mechanisms.

“Collagen” as described herein is the main structural protein in the extracellular space in the various connective tissues in the body. Collagen forms fibrils and sheets that bear tensile loads. Collagen also has specific integrin-binding sites for cell adhesion and is known to promote cell attachment, migration, and proliferation. Collagen may be positively charged because of its high content of basic amino acid residues such as arginine, lysine, and hydroxylysine. Over 90% of the collagen in the human body is Type I collagen. Type III collagen is the main component of reticular fibers and may be commonly found alongside Type I collagen. One of skill in the art would appreciate that the collagen may be provided from a commercial source. In some embodiments of any one of each or any of the above- or below- mentioned embodiments, the collagen material provided may have a mixture of about 97% to about 99% collagen Type I with the remaining collagen being about 1% to 3% collagen Type III.

In some embodiments of any one of each or any of the above- or below-mentioned embodiments, the collagen is crosslinked collagen. In some embodiments of any one of each or any of the above- or below-mentioned embodiments, the collagen is non-crosslinked collagen.

In some embodiments of any one of each or any of the above- or below-mentioned embodiments, HA is crosslinked to an amine and may have more than one crosslink through a lysine on either the collagen or HA, or by another amine group.

“Elastic modulus” is also known as the modulus of elasticity and refers to a quantity that measures an object or substance's resistance to being deformed elastically (i.e., non-permanently) when a stress is applied to it.

“Compression force,” as described herein refers to the application of power, pressure, or exertion against an object that causes it to become squeezed, squashed, or compacted.

“Sterilization” as described herein, refers to submission of a material to a sterilization process which may lead to death of microorganisms in the material. Methods for disinfection and sterilization may be by physical, chemical and physicochemical means.

For materials such as hydrogels, sterilization may be accomplished with less aggressive conditions, such as less time for sterilization, lower temperature and lower dose exposure, for example.

Without being limiting, sterilization may include steam heat, dry heat, and/or ionizing radiation

A sterile product, such as a hydrogel may be formed as the result of being subjected to a sterilization process. Such sterilization processes may be found in Chitre et al., (US 2014/0011980 A1), Chitre et al. (US 2018/0147307 A1)), and Chitre et al. (US 2016/0101200 A1).

In an embodiment, the composition or matrix comprises an anesthetic. Without being limiting anesthetics include, benzocaine, choloroprocaine, procaine, proparacaine, tetracaine, amylocaine, oxybuprocaine, articaine, bupivacaine, dibucaine, etidocaine, levobupivacaine, lidocaine, mepivacaine, prilocaine, ropivacaine, sameridine, tonicaine, cinchocaine.

### Methods

Hyaluronic acid and collagen may be co-crosslinked using 1-ethyl-3-(N,N′-dimethylaminopropyl)carbodiimide (EDC) and N-hydroxysuccinimide (NETS) to activate native carboxylic acid moieties present on the HA and collagen towards reaction with endogenous amine groups present on the collagen. In an embodiment, lysine is added as an additional di-amine crosslinker to further enhance the HA and collagen chemical modification and to tune the physical properties of the resulting hydrogels.

The addition of lysine may allow for an independent adjustment of the crosslinking may modulate the hydrogel physical properties without altering the HA:collagen composition or the amount of activating reagent. In an embodiment, the crosslinking reaction is run under mild pH and temperature conditions (pH 5.5 and 4-25° C.), rather than the higher pH and temperature required for BDDE crosslinking which is standard for HA dermal fillers. Using the methods, surprisingly the HA and sensitive collagen protein components were shown to undergo minimal degradation during crosslinking and their structure remains largely intact, as shown in the Examples.

In an embodiment the hyaluronic acid is hydrated for at least 60 minutes prior to the crosslinking step with collagen. In a further embodiment, the hyaluronic acid is hydrated at a temperature below room temperature. In a still further embodiment, the hyaluronic acid is hydrated at a temperature of about 2° C., about 4° C., about 6° C., about 8° C., about 10° C., about 12° C., about 14° C., about 16° C., about 20° C., about 22° C., about 24° C. or any temperature in between a range defined by any two aforementioned values. In an embodiment, the hyaluronic acid is hydrated at a temperature above room temperature. Thus, the methods of making the hydrogel may be tuned in order to tune the hydrogel properties such as the tan del parameter (G″/G′), for example.

In an embodiment, the collagen may be provided in a solution, wherein the solution comprises an acidic pH, wherein the collagen is soluble.

In an embodiment, the collagen is provided as pre-fibrillated collagen, wherein the collagen is treated prior to crosslinking. In an embodiment, the pre-fibrillated collagen is within a basic solution. In an embodiment, the collagen is provided as a soluble collagen, wherein the collagen is in an acidic solution. In an embodiment, the pre-fibrillated collagen is within solution, wherein the solution comprises a neutral pH.

In an embodiment, the crosslinking reaction is run at a pH of 4.0, 5.0, 5.5, 6.0, 6.5 or 7.0 or any pH in between a range defined by any two aforementioned values.

Hydrogel physical properties may be dependent on HA and collagen concentrations, HA molecular weight, EDC concentration, EDC/NHS ratio, temperature, pH, salt/buffer concentration, and lysine concentration. In an embodiment, elastic modulus (G′) values ranging from 30 Pa to almost 10,000 Pa are obtained. In an embodiment, the elastic modulus depends on the formulation and synthesis parameters. The formulations and synthesis parameters may be adjusted. In an embodiment, the compression force values ranges from 20 gmf to more than 500 gmf and the hydrogel swelling can range from 1.5 times to 5 times the original gel volume. Based on the broad spectrum of obtained physical properties, different HA/collagen configurations might find use as dermal fillers for different facial applications.

In an embodiment of the crosslinking reaction, the method comprises stopping the crosslinking step.

As described in the embodiments herein, formulations with low G′ and compression force values and minimal swelling may have applications for very superficially-placed fine-line fillers or injectable skin quality enhancers whereas more robust configurations with higher G′ and compression force and more swelling could be used for moderate to severe wrinkle correction and facial volumizing/contouring.

In an embodiment, methods of filling fine lines comprise the steps of providing a patient in need a composition comprising a low G′ and compression force values. In an embodiment, methods of treating moderate to severe wrinkle correction and facial volumizing/contouring is provided, the method comprising providing a patient in need a composition comprises a higher G′ and compression force.

Increasing the HA concentration of a crosslinked macromolecular matrix is contemplated. Increasing the HA concentration may result in hydrogels with higher G′, higher compression force values and higher opacity, for example. An increase in opacity may also decrease the possibility of a blue discoloration in the site of injection, which is due to the Tyndall effect. A strong crosslinked macromolecular matrix may provide a force that is required to lift the tissue and resist subsequent deformation, which may result in the desired correction and appearance. Thus a high lifting capacity may require a high strength of the matrix. The elastic modulus (G′) may represent the stiffness of the matrix and the ease of extrusion of the matrix.

The elastic modulus may be a function of the concentration of the hyaluronic acid. In an embodiment, the HA concentration is in between a range of 13 mg/ml to 28 mg/ml. In an embodiment, the composition comprises a G′ of about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, about 3000 Pa, about 3100 Pa, about 3200 Pa, about 3300 Pa, about 3400 Pa, about 3500 Pa, about 3600 Pa, about 3700 Pa, about 3800 Pa, about 3900 Pa, about 4000 Pa, about 4100 Pa, about 4200 Pa, about 4300 Pa, about 4400 Pa, about 4500 Pa, about 4600 Pa, about 4700 Pa, about 4800 Pa, about 4900 Pa, about 5000 Pa, about 5100 Pa, about 5200 Pa, about 5300 Pa, about 5400 Pa, about 5500 Pa, about 5600 Pa, about 5700 Pa, about 5800 Pa, about 5900 Pa, about 6000 Pa, about 6100 Pa, about 6200 Pa, about 6300 Pa, about 6400 Pa, about 6500 Pa, about 6600 Pa, about 6700 Pa, about 6800 Pa, about 6900 Pa, about 7000 Pa, about 7100 Pa, about 7200 Pa, about 7300 Pa, about 7400 Pa, about 7500 Pa, about 7600 Pa, about 7700 Pa, about 7800 Pa, about 7900 Pa, about 8000 Pa, about 8100 Pa, about 8200 Pa, about 8300 Pa, about 8400 Pa, about 8500 Pa, about 8600 Pa, about 8700 Pa, about 8800 Pa, about 8900 Pa, about 9000 Pa, about 9100 Pa, about 9200 Pa, about 9300 Pa, about 9400 Pa, about 9500 Pa, about 9600 Pa, about 9700 Pa, about 9800 Pa, about 9900 Pa, or about 10,000 Pa or any elastic modulus in between a range defined by any two aforementioned values.

In an embodiment, a higher G′ value is desired. Higher G′ values may also be obtained by increasing the EDC:HA ratio or the EDC:NHS ratio. A mixture of hyaluronic acid components comprising different molecular weights is contemplated, which may affect G′ and compression force values. For example, in a HA:collagen formulation, a decreased hydration temperature may result in a higher G′ value, lower swelling, and decreased opacity (increased translucency). Using these synthesis parameters and results, HA-collagen formulations with the desired physical properties may be targeted and synthesized.

Collagen concentrations may also affect the physical properties. For a given hydration temperature, increased collagen concentrations may result in increased opacity, higher G′, and decreased swelling. The increased opacity may lead to a decrease in a Tyndall effect of a filler. The physical and optical properties of the HA-Collagen hydrogel are dependent on the degree to which the collagen is soluble during the synthesis steps. Synthesis parameters such as temperature, pH, and salt concentration affect collagen solubility. Collagen solubility may be decreased by increased temperature, pH, and salt concentration and decreased collagen solubility during synthesis results in gels with lower G′, higher swelling, and increased extrusion force. HA may also interact with collagen to decrease collagen solubility as described by Taguchi and coworkers (Taguchi et al. Journal of Biomedical Materials Research, 2002, 61(2), 330-336; incorporated by reference herein). Modulating the salt concentration may alter the interaction between the HA and collagen, thereby adjusting the collagen solubility and changing the physical properties. In an embodiment, the composition comprises a salt comprising a concentration of between 50 mM to 400 mM. In an embodiment, the composition comprises a NaCl concentration of about 150 mM. Therefore, maximal G′ and minimal swelling values may be obtained with maximal collagen solubility for a given HA concentration during synthesis by decreasing the hydration temperature and optimizing the salt/buffer concentrations.

In an embodiment, the composition is transparent. In an embodiment, the composition is translucent. In an embodiment, the concentration of HA, hydration temperature, salt concentration and/or collagen concentration affects the composition opacity. The increased opacity may reduce the Tyndall effect, the blue discoloration that may be seen at an injection site. Methods of measuring the opacity of a composition may be appreciated by one of skill in the art.

In an embodiment, the biological properties and tissue response to these materials and compositions have been characterized. In an embodiment, the formulations show enhanced cell activity compared to HA-only materials. The activity level is dependent on the collagen concentration, but also on HA concentration and synthesis procedure. In an embodiment, formulations with lower HA concentrations (13 mg/mL) were found to give an enhanced in vitro response over those with higher HA concentrations (20-28 mg/mL) for a given collagen concentration. In an embodiment, compositions with similar HA concentrations, such as those hydrogels with higher collagen concentrations showed a greater in vitro response. Additionally, in an embodiment, formulations which were hydrated at temperatures below room temperature stimulated higher cell activity than those similar formulations which were hydrated at room temperature. A certain cell activity level may be desired for particular filler indication and by selecting the formulation and synthesis parameters, the preferred activity level could be achieved.

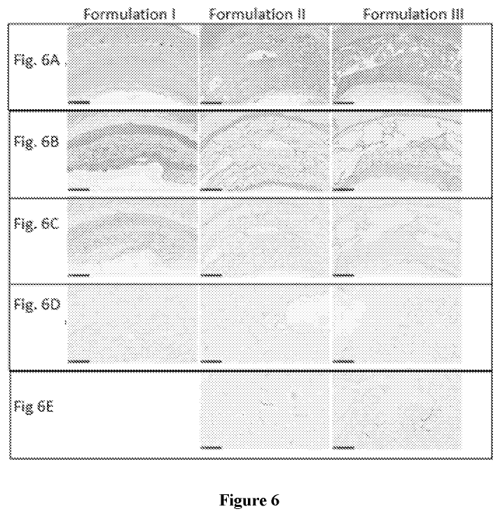

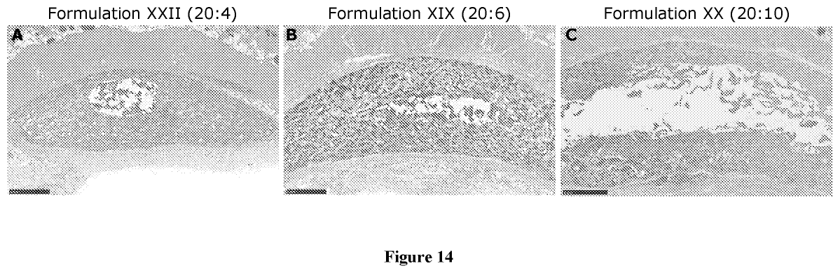

In an embodiment, HA/collagen formulations were also evaluated for tissue response in a tissue integration model. Tissue sections from implants of HA-Collagen materials showed cellular infiltration from the surrounding tissue as well as new collagen deposition and blood vessel formation within the injected filler bolus. The degree of infiltration and tissue integration differed between different formulations. In some embodiments of the formulations described herein, collagen structure and crosslinking is surprisingly important in the degree of infiltration and tissue integration. In an embodiment, collagen structure and crosslinking may be important in tissue integration and infiltration (see, e.g., FIG. 14). In some embodiments of the formulations described herein, the tissue integration decrease with increasing HA concentrations. In some embodiments of the formulations described herein, formulations with low HA concentrations (13 mg/ml) that were injected into tissue, the surrounding tissue was found to infiltrate the entire gel bolus by 4 weeks. In these embodiments, cell nuclei and newly deposited collagen were found to be interspersed among the gel. This may be seen in Example 7 (FIGS. 6B and 6C) using Formulation I. Thus, these formulations led to the surprising result of tissue infiltration into the gel bolus.

In some embodiments, formulations with higher HA concentrations (20-25 mg/mL) also demonstrated strong tissue integration, but the tissue did not infiltrate the entire bolus as for those formulations with lower HA concentrations.

In some embodiments, HA molecular weight and/or gel particle properties also influence surrounding tissue integration.

However, another surprising outcome showed that structure/crosslinking may be more important than collagen concentration in a composition. One example of this surprising finding is that a gel with 20:6 HA:Collagen, which had HA that was hydrated at 5° C. demonstrated a better tissue integration score (score=2.0) than a gel with a 20:10 HA:Collagen that was hydrated at room temperature (score=0.5). The tissue integration scoring was performed by a blinded histopathologist and normalized to the study internal control (HA only gel) A higher score indicates better tissue integration.

Surprisingly, a difference in outcome was shown in compositions that differed in the structure of the mixed gels. Compositions in which the collagen was mixed in, had a different response than compositions in which the gels had collagen that were crosslinked to HA at 5° C.

Formulation XIX synthesized with HA hydrated at 5° C. demonstrated improved in vitro and in vivo performance as well as the best tissue integration as shown in the embodiments herein.

Aside from the collagen concentration, the level or crosslinking and the structure of the composition showed equal importance. For example, compositions such as gel formulations produced at reduced temperatures resulted in improved in vitro and in vivo performance, such as improved tissue integration. This may be seen for Formulation XIX, which had a 20:6 HA:Collagen ratio and a hydration temperature of about 5° C. Preparation of the formulation also led to surprising outcomes such as improvement of in vitro and in vivo performance. In some embodiments, the preparation of the gel formulation at a low temperature for hydration, for example, 5° C. led to gel formulation with improved in vitro and in vivo performance. In some embodiments, the gel formulation demonstrated tissue integration into the site of the injected formulation.

Formulations with a 20:6 and a 20:4 HA:Collagen ratio and a hydration temperature of about 5° C. also led to surprising outcomes such as improvement of in vitro and in vivo performance.

In some embodiments, a formulation is provided, wherein the formulation increases infiltration of collagen into a tissue. The formulation comprises 13 mg/ml hyaluronic acid. In some embodiments, the formulation is injected into a tissue, thus creating a depot comprising the formulation, wherein cells from the tissue surrounding the depot is deposited into the depot. In an embodiment, the tissue injected by the formulation is shown to have tissue integration and collagen deposition and blood vessel formation. In an embodiment, the formulation comprises a 20:6 HA:Collagen ratio and a hydration temperature of about 5° C. In an embodiment, the formulation comprises a 20:4 HA:Collagen ratio and a hydration temperature of about 5° C.

A primary function of dermal fillers is to fill wrinkles and support the overlying tissue under which they were injected. The amount of lift required is dependent on the particular facial indication. Products intended for volumizing indications and placed deeper under the skin will need to exhibit more structure and more lift. Formulations for fine lines with superficial placement need not demonstrate as much lift but should be smoother and blend into the existing tissue. Therefore, HA/collagen formulations were assessed in an animal lift capacity model to determine lift capacity. For formulations which were crosslinked in a similar way, the lift capacity was dependent on the HA concentration with higher HA concentrations providing increased lift.

In some embodiments, crosslinked HA:Collagen formulations with added lysine demonstrated an increase in lift as the HA concentration was increased from 13 mg/mL to 20 mg/mL to 25 mg/mL. In some embodiments, the HA molecular weight also affects lift. In some embodiments, a formulation containing 25 mg/mL of high molecular weight HA demonstrated greater lift than a formulation composed of 25 mg/mL of a mixture of low and high molecular weight HA. Thus, the desired lift can be achieved by selecting the optimal synthesis parameters, HA concentration, and HA molecular weight ratio. In some embodiments of any one of each or any of the above- or below-mentioned embodiments, the formulation comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

### Methods of Synthesizing a Lysine-Crosslinked HA-Collagen Hydrogel

The present disclosure provides methods comprising providing a collagen solution and adding the collagen solution to a second solution comprising lysine⋅HCl, high molecular weight HA, MES buffer, NaCl and NaOH. In some embodiments, the hydrogel comprises a weight ratio of hyaluronic acid to collagen at about 24:12, about 28:2, about 20:4, about 25:4, about 22:6, about 22:4, about 24:6, about 20:6, or about 13:4. In some embodiments, the hydrogel comprises a concentration of collagen of about 6 mg/ml. In some embodiments, the hydrogel is stirred for homogenization. In some embodiments, the hydrogel is hydrated below room temperature. In an embodiment, the HA is hydrated at a temperature between 2° C. and 35° C. In an embodiment, the HA is hydrated at a temperature of 2° C., 3° C., 5° C., 7° C., 9° C., 11° C., 13° C., 15° C., 17° C., 19° C., 21° C., 23° C., 25° C., 27° C., 29° C., 31° C., 33° C., 35° C., or any temperature in between a range defined by any two aforementioned values. In an embodiment, the HA is hydrated at a temperature between 2° C. and 19° C. In an embodiment, the HA is hydrated at a temperature of 2° C., 3° C., 5° C., 7° C., 9° C., 11° C., 13° C., 15° C., 17° C., or 19° C. or any temperature in between a range defined by any two aforementioned values. In an embodiment, the HA is hydrated for at least 60 minutes at room temperature. In an embodiment, the HA is hydrated at a temperature above room temperature. In an embodiment, the HA is hydrated at a temperature of at least 35° C. In some embodiments, another hydration step is performed for at least 60 minutes. In some embodiments, the hydration is performed in MES buffer at about a pH of 5.5. The mixture may be contained within a syringe and may be passed between a syringe at least fifty times between two syringes. A solution of EDC/NHS may be added to the mixture. Mixing may be performed by passing the solution in between two syringes. After the EDC/NHS solution is added, the mixture is allowed to react for at least 16 hours at a temperature between 2-8° C. In some embodiments, the pH of the solution is adjusted to 7.4 using NaOH and purified using dialysis. The properties of the formed hydrogel may be obtained using a rheometer. One of skill in the art may measure the composition for several parameters such as compression force, swelling characteristics, and extrusion force, for example.

In an embodiment, the macromolecular matrix further comprises un-crosslinked HA, which may be used to ease injection and decrease the extrusion force.

In an embodiment, the lysine:HA ratio is optimized to maximize the crosslinking efficiency. In an embodiment , lysine:HA ratio is between about 0.0 to 0.5 and may allow for more efficient crosslinking. Crosslinking without lysine may rely on collagen to provide the amines for crosslinking and may allow for more water-labile ester crosslinks between HA chains. Crosslinking with high lysine:HA ratios may saturate the activated carboxylic acids on the HA chains and may lead to pendant lysine molecules attached to the HA chain on one side only, rather than crosslinking between chains. By selecting the optimal lysine:HA ratio for a given indication, the physical properties of the resulting hydrogel can be tuned and the desired characteristics can be achieved. In some embodiments, the optimal lysine:HA ratio may be composition dependent.

### Sterilization of the Compositions

Developed biomaterials may require sterilization, or the destruction of unwanted biologic material such as pathogens, microbes of bacteria, and prior to the administration of the composition by injection or implantation into a human patient. These compositions include the embodiments described herein, for example the materials such as crosslinked macromolecular matrix. Proteins, polysaccharides and carbohydrates in these materials may be susceptible to molecular breakdown when exposed to conventional heat temperature sterilization procedures, such as autoclave, or when subjected to ionizing radiation such as gamma radiation. Conventionally, many of these energy-sensitive biomaterials are sterilized in bulk by microfiltration processes which are intended to physically remove microbes from the compositions. The filtered compositions must then be packaged in syringes and/or vials for use by physicians.

In an embodiment, the crosslinked macromolecular matrix is sterile. In an embodiment, the methods of making the crosslinked macromolecular matrix further comprise a step of sterilizing the crosslinked macromolecular matrix.

In an embodiment, the methods further comprise the step of subjecting the composition or crosslinked macromolecular matrix to a dose of broadband spectrum radiation effective to inactivate pathogen, microbes and other microorganisms.

In an embodiment, the methods further comprise the step of subjecting the composition or crosslinked macromolecular matrix to pulsed radiation, hereinafter sometimes pulsed light, comprising broadband spectrum radiation. The broadband spectrum radiation may have a band range from about 100 nm to about 1100 nm wavelength. The broadband spectrum radiation includes wavelengths in the ultraviolet range, the visible light range and the infrared range. In some embodiments, has a wavelength distribution of about 54% UV wavelengths, 26% visible wavelengths and about 20% infrared wavelengths. This form of radiation may be provided by a Xenon lamp.

In an embodiment, the pulsed light inactivates microorganisms and microbes in the composition, throughout the composition, without causing significant deterioration of the composition, and without causing significant change in rheology of the composition.

In an embodiment, the pulsed light has an energy defined by a UV fluence at 254 nm of between about 100 mJ/sqcm to about 2000 mJ/sqcm. In an embodiment, the pulsed light has an energy defined by a UV fluence at 254 nm of between about 300 mJ/sqcm to about 1800 mJ/sqcm.

In an embodiment, the pulsed light has an energy defined by a UV fluence at 254 nm of between about 700 mJ/sqcm to about 800 mJ/sqcm. In an embodiment, the pulsed light has an energy defined by a UV fluence at 254 nm of between about 1400 mJ/sqcm to about 1600 mJ/sqcm.

In an embodiment, the pulsed light has a pulse frequency of between about 1 pulse per second to about 10 pulses per second, for example, about 3 pulses per second.

In an embodiment, the composition is subjected to the pulsed light for a time period of no greater than 240 seconds. In one embodiment, the composition is subjected to the pulsed light for a time period of no greater than 120 seconds. In an embodiment, the composition is subjected to the pulsed light for a time period of no greater than 40 seconds In an embodiment, the composition is subjected to the pulsed light for a time period of no greater than 30 seconds. In one embodiment, the composition is subjected to the pulsed light for a time period of no greater than 20 seconds. In an embodiment, the composition is subjected to the pulsed light for a time period of 10 seconds.

In an embodiment, the composition is subjected to the pulsed light for a time period of 5 seconds. In an embodiment, the composition is subjected to the pulsed light for a time period of no greater than one second.

In an embodiment, the pulsed light is effective to sterilize the composition without raising the temperature of the composition more than 90 degrees C. In an embodiment, the pulsed light is effective to sterilize the composition without raising the temperature of the composition more than 20 degrees C. In an embodiment, the dose is effective to sterilize the composition without raising the temperature of the composition more than 15° C., for example, more than 10° C., for example, more than 5° C.

In an embodiment, the pulsed light is effective to sterilize the composition with a loss in rheology (G′/G″) of less than about 10%, or less than about 8%, or less than about 5%.

In an embodiment, the pulsed light is effective to sterilize the composition, that is, inactivate pathogens, microbes and other microorganisms in the composition, without causing significant deterioration, for example, without causing significant changes in rheological properties of the composition.

In an embodiment, the effective sterilizing dose of the radiation retains the rheology of the hydrogel. In an embodiment, the methods are effective to sterilize the hydrogel with a loss in rheology (G′/G″) of less than about 10%, or less than about 8%, or less than about 5%.

### EXAMPLES

The following examples, including the experiments conducted and the results achieved, are provided for illustrative purposes only and are not to be construed as limiting the disclosure.

### Example 1

### Synthesis of a Lysine-Crosslinked HA-Collagen Hydrogels

A 4.96 mg/mL collagen solution in 0.01M HCl was added to a 30 mL HSW Norm-Ject syringe along with lysine⋅HCl, BMW HA, MES buffer/NaCl solid and 1M NaOH. Concentrations were adjusted accordingly to make hydrogels with, e.g., an HA:Collagen ratio of 13:4 mg/ml (Formulation I), 20:4 mg/ml (Formulation II) and 25:4 (Formulation III). The mixture was stirred to homogenize the solution and the HA was allowed to hydrate for approximately 60 minutes at room temperature. After about 60 minutes to about 90 minutes, the mixture was passed between syringes and allowed to hydrate again for about 30 minutes to about 60 minutes. After the second hydration, the mixture was passed between syringes several times. An EDC/NHS solution was prepared in a third 30 mL syringe by adding water, NHS, and EDC, and was shaken to mix. The EDC/NHS solution was added to the HA/collagen mixture and passed between two syringes before transferring to glass vials which were allowed to react at 2-8° C. In some embodiments, the reaction time is about 16 hours, about 18 hours, about 20 hours, about 22 hours, about 24 hours or any time in between a range defined by any two aforementioned values. After this time, the gel was transferred to syringes and again passed between two syringes. The pH of the gel was adjusted to approximately 7.40 using 2M NaOH and the final volume was adjusted using PBS. The gel formulation was dialyzed against PBS at 2-8° C. for approximately 70 hours with several buffer changes during that time to remove the EDC/NHS. The gel was then transferred from the dialysis membrane to syringes and passed through a stainless steel mesh (60 μm pores-104 μm pores) and passed between two syringes. The gel was transferred to 1 mL syringes and the syringes were steam sterilized. The resulting sterile hydrogel was characterized using rheology, compression force measurements, extrusion force measurements, and swelling.

For Formulation XXVI, NaCl was omitted during crosslinking.

For Formulations XXV and XXVI, uncrosslinked BMW HA (2% (w/w) relative to the total composition) and Lidocaine HCl (0.3% (w/w) relative to the total composition) is added prior to syringe filling and sterilization.

### Example 2

### Synthesis of a Lysine-Crosslinked HA-Collagen Hydrogel with a Final Collagen Concentration of 6 mg/mL

A 7.16 mg/mL collagen solution in 0.01M HCl was added to a 30 mL HSW Norm-Ject syringe along with lysine⋅HCl, BMW and/or LMW HA and a MES buffer NaCl solid. The pH was adjusted with 1M NaOH. The mixture was stirred to homogenize and the HA was allowed to hydrate for approximately 60 minutes at the specified temperature. After about 60-90 minutes, the mixture was passed several times between two syringes and allowed to hydrate again for at least 30 minutes. After the second hydration step, the mixture was again passed between two syringes several times. An EDC/NHS solution was prepared in a third 30 mL syringe by adding water, NHS, and EDC and was shaken to mix. Hydration may be performed at a temperature of about 5° C., about 6° C., about 7° C., about 8° C., about 9° C., about 10° C., or any temperature in between a range defined by any two aforementioned values. The EDC/NHS solution was added to the HA/collagen mixture and passed between two syringes several times before transferring to a Thinky Mixer reaction vessel which were allowed to react at 2-8° C. for at least 16 hours. After this time, the gel was homogenized using the Thinky Mixer. The pH of the gel was adjusted to approximately 7.40 using 2M NaOH and the final volume was adjusted using PBS. The gel formulation was dialyzed against PBS at 2-8° C. for approximately 70 hours several buffer changes during that time. The gel was then transferred from the dialysis membrane to syringes and passed through a stainless steel mesh (104 μm pores) and homogenized using the Thinky Mixer. The gel was transferred to 1 mL syringes and the syringes were steam sterilized. The resulting sterile hydrogel was characterized as described in the Examples above.

In some embodiments, the gel comprises 20 mg/ml hyaluronic acid. In some embodiments, the gel comprises 6 mg/ml collagen. In some embodiments of the method of making the gel, the hyaluronic acid is hydrated at a temperature of 5° C.

### Example 3

### Synthesis of a Lysine-Crosslinked HA-Collagen Hydrogel with HA:Collagen Concentrations of 28:2 mg/mL (Formulation XVI)

A 3.20 mg/mL collagen solution in 0.01M HCl was added to a 30 mL HSW Norm-Ject syringe along with 0.01M HCl, lysine⋅HCl, HMW HA, LMW HA and MES buffer/NaCl solid. The pH was adjusted using NaOH. The mixture was stirred to homogenize and the HA was allowed to hydrate for approximately 90 minutes at room temperature. After 90 minutes, the mixture was passed several times between syringes and allowed to hydrate again for approximately 30 minutes. After the second hydration step, the mixture was passed between syringes several times. An EDC/NHS solution was prepared in a third 30 mL syringe by adding water, NHS, and EDC and was shaken to mix. The EDC/NHS solution was added to the HA/collagen mixture and passed between syringes several times before to transferring to glass vials which were allowed to react at 2-8° C. for at least 16 hours. After this time, the gel was transferred to syringes and passed between syringes. The pH of the gel was adjusted to approximately 7.40 using 2M NaOH and the final volume was adjusted using PBS. The gel formulation was dialyzed against PBS at 2-8° C. for approximately 70 hours with several buffer changes during that time. The gel was then transferred from the dialysis membrane to syringes and passed through a stainless steel mesh (104 μm pores) and passed between syringes to homogenize. The gel was transferred to 1 mL syringes and the syringes were steam sterilized. The resulting sterile hydrogel was characterized as described in the Examples above.

### Example 4

### Synthesis of a Lysine-Crosslinked HA-Collagen Hydrogel with HA:Collagen Concentrations of 25:4 mg/mL Prepared at 1.25× of the Final Concentration (Formulation XV)

A 5.67 mg/mL collagen solution in 0.01M HCl was added to a 30 mL HSW Norm-Ject syringe along with lysine⋅HCl, HMW HA, LMW HA and MES buffer/NaCl solid. The pH was adjusted with 1M NaOH. The mixture was stirred to homogenize and the HA was allowed to hydrate for approximately 90 minutes at room temperature. The mixture was then passed several times between syringes and allowed to hydrate again for 30 minutes. After the second hydration step, the mixture was again passed between syringes. An EDC/NHS solution was prepared in a third 30 mL syringe by adding water, NHS, and EDC and was shaken to mix. The EDC/NHS solution was added to the HA/collagen mixture and passed between syringes several times before transferring to glass vials which were allowed to react at 2-8° C. for at least 16 hours. After this time, the gel was transferred to syringes and passed between syringes. The pH of the gel was adjusted to approximately 7.40 using 2M NaOH and the final volume was adjusted using PBS. The gel formulation was dialyzed against PBS at 2-8° C. for approximately 70 hours with several buffer changes during that time. The gel was then transferred from the dialysis membrane to syringes and passed through a stainless steel mesh (104 μm pores) and passed between syringes to homogenize. The gel was transferred to 1 mL syringes and the syringes were steam sterilized. The resulting sterile hydrogel was characterized as described in the Examples above.

### Example 5

### Physical Properties of Hydrogels

The rheological properties were obtained using an Anton-Paar MCR301/302 rheometer with a 25 mm parallel plate geometry measuring tool. The samples were analyzed at a 1 mm gap height with both frequency sweep (10 Hz-0.1 Hz, 1% strain) and amplitude sweep (0.3%-300% strain, 5 Hz frequency) measurements. Compression force was measured using the same instrumentation with a gap height of 2.5 mm and a vertical compression. The gap height was established at 2.5 mm and remained there for 5 minutes, then compressed from 2.5 mm to 0.89 mm with a velocity of 13.33 μm/s. Hydrogel swelling was measured by mixing a gel sample with excess phosphate buffer and determining the volume of the gel after equilibrium. The swollen gel volume was compared back to original gel volume added before the buffer addition. The swelling is expressed as additional fluid uptake as a percentage of the original gel volume. The gel extrusion force was measured for gel formulations in 1 mL COC syringes fitted with ½ 27G TSK needles (unless otherwise stated) using a Texture Analyzer set at a speed of 50 mm/min.

TABLE 1
Hydrogel formulation synthesis parameters and physical properties. 1
Eq corresponds to an MES buffer concentration of 0.1M with 0.9% NaCl.

| | HA | Collagen | HMW/ | Hydrat. | | Lysine:HA | EDC:HA | Buffer/ | |
|---|---|---|---|---|---|---|---|---|---|
| | Conc. | Conc. | LMW HA | Temp | | Mole | Mole | Salt | |
| Sample | mg/mL | mg/mL | Ratio | (° C.) | | Ratio | Ratio | Conc. | |
| 13:4, Form. I | 13 | 4 | 100/0 | 22 | | 0.339 | 1.235 | 1 | Eq |
| 20:4, Form. II | 20 | 4 | 100/0 | 22 | | 0.166 | 1.003 | 1 | Eq |
| 25:4, Form. III | 25 | 4 | 100/0 | 22 | | 0.166 | 1.003 | 1 | Eq |
| 20:4, Form. IV | 20 | 4 | 100/0 | 22 | | 0.166 | 1.003 | 1 | Eq |
| 20:6, Form. V | 20 | 6 | 100/0 | 22 | | 0.166 | 1.003 | 1 | Eq |
| 24:6, Form. VI | 24 | 6 | 100/0 | 22 | | 0.167 | 1.003 | 1 | Eq |
| 24:6, Form. VII | 24 | 6 | 65/35 | 22 | | 0.167 | 1.003 | 1 | Eq |
| 24:6, Form. VIII | 24 | 6 | 35/65 | 22 | | 0.167 | 1.003 | 1 | Eq |
| 24:6, Form. IX | 24 | 6 | 100/0 | 15 | | 0.167 | 1.003 | 1 | Eq |
| 24:6, Form. X | 24 | 6 | 100/0 | 5 | | 0.167 | 1.003 | 1 | Eq |
| 24:6, Form. XI | 24 | 6 | 100/0 | 35 | | 0.167 | 1.003 | 1 | Eq |
| 24:6, Form. XII | 24 | 6 | 100/0 | 5 | | 0.167 | 1.003 | 0.33 | Eq |
| 24:6, Form. XIII | 24 | 6 | 100/0 | 22 | | 0.167 | 1.003 | 0.33 | Eq |
| 24:6, Form. XIV | 24 | 6 | 100/0 | 35 | | 0.167 | 1.003 | 0.33 | Eq |
| 25:4, Form. XV | 25 | 4 | 10/90 | 22 | | 0.334 | 1.250 | 1 | Eq |
| 28:2, Form. XVI | 28 | 2 | 10/90 | 22 | | 0.333 | 1.000 | 1 | Eq |
| 28:2, Form. XVII | 28 | 2 | 10/90 | 22 | | 0.500 | 1.000 | 1 | Eq |
| 28:2, Form. XVIII | 28 | 2 | 10/90 | 22 | | 0.000 | 1.000 | 1 | Eq |
| 20:6, Form. XIX | 20 | 6 | 100/0 | 5 | | 0.166 | 1.003 | 1 | Eq |
| 24:10 Form. XX | 20 | 10 | 100/0 | 25° | C. | 0.320 | 0.855 | 1 | Eq |
| 24:6 Form. XXI | 24 | 6 | 100/0 | 25° | C. | 0.320 | 0.855 | 1 | Eq |
| 20:4; Form. XXII | 20 | 4 | 90/10 | 5° | C. | 0.166 | 1.000 | 1 | Eq |
| 22:4 Form. XXIII | 22 | 4 | 95/5 | 5° | C. | 0.166 | 1.000 | 1 | Eq |
| 25:6.8 Form. XXIV | 25 | 6.8 | 90/10 | 5° | C. | 0.166 | 1.000 | 1 | Eq |
| 20:4 Form. XXV | 20 | 3.6 | 90/10 | 5° | C. | 0.166 | 1.000 | 1 | Eq |
| 20:4 Form. XXVI | 20 | 3.6 | 90/10 | 5° | C. | 0.166 | 1.000 | 1 | Eq |

| | | | | Compress. | | | |
|---|---|---|---|---|---|---|---|
| | G′ | G″ | G″/ | Force | | Extrusion | |
| Sample | Pa | Pa | G′ | (gmf) | Swelling | Force (N) | |
| 13:4, Form. I | 380 | 46.4 | 0.123 | 47 | 129% | 13.8 | (30 G) |
| 20:4, Form. II | 645 | 66.0 | 0.103 | 180 | 237% | 28.0 | |
| 25:4, Form. III | 1370 | 76.6 | 0.056 | 310 | 236% | 51.7 | |
| 20:4, Form. IV | 860 | 65.6 | 0.076 | 190 | 243% | 32.4 | |
| 20:6, Form. V | 1265 | 94.7 | 0.075 | 174 | 194% | 15.3 | |
| 24:6, Form. VI | 1360 | 74.3 | 0.055 | 292 | 238% | 63.6 | |
| 24:6, Form. VII | 1180 | 79.7 | 0.068 | 226 | 230% | 29.2 | |
| 24:6, Form. VIII | 932 | 79.2 | 0.085 | 163 | 241% | 20.0 | |
| 24:6, Form. IX | 3145 | 192 | 0.061 | 341 | 159% | 33.8 | |
| 24:6, Form. X | 4750 | 470 | 0.099 | 323 | 127% | 20.1 | |
| 24:6, Form. XI | 876 | 68.0 | 0.078 | 247 | 257% | 56.1 | |
| 24:6, Form. XII | 5840 | 738 | 0.126 | 345 | 99% | 19.2 | |
| 24:6, Form. XIII | 5470 | 612 | 0.112 | 334 | 108% | 20.5 | |
| 24:6, Form. XIV | 2835 | 165 | 0.058 | 274 | 168% | 30.2 | |
| 25:4, Form. XV | 1015 | 83 | 0.082 | 171 | 172% | 24.5 | |
| 28:2, Form. XVI | 578 | 86.6 | 0.149 | 166 | 288% | 23.4 | |
| 28:2, Form. XVII | 356 | 91.8 | 0.258 | 122 | 418% | 16.2 | |
| 28:2, Form. XVIII | 538 | 94.1 | 0.175 | 144 | 341% | 21.4 | |
| 20:6, Form. XIX | 3395 | 352 | 0.104 | 251 | 120% | 15.4 | |
| 24:10 Form. XX | 1183.3 | 126.6 | 0.107 | 229 | 124 | 74.28 | (30 G) |
| 24:6 Form. XXI | 1013.3 | 100.3 | 0.099 | 192 | 117 | 81.49 | (30 G) |
| 20:4; Form. XXII | 2842.7 | 276.3 | 0.097 | 136 | 15 | 37.21 | (30 G) |
| 22:4 Form. XXIII | 3776.3 | 350.2 | 0.093 | 192 | 28 | 37.74 | (30 G) |
| 25:6.8 Form. XXIV | 8225.7 | 1932.3 | 0.229 | 167 | — | 25.98 | (30 G) |
| 20:4 Form. XXV | 1500.5 | 157.9 | 0.105 | 109 | — | 10.7 | (30 G) |
| 20:4 Form. XXVI | 2857.0 | 398.1 | 0.140 | 117 | — | 12 | (30 G) |

As shown in Table 1, as the HA concentration increases from 13 to 20 to 25 mg/mL with constant collagen concentration (Formulation I vs. Formulation II vs. Formulation III), the G′ value increases (380→645→1370 Pa) along with compression force (47→180→310 gmf) and extrusion force (13.8 (30G)→28.0→51.7 N) whereas G″/G′ ratio decreases with increasing HA concentration (0.123→0.103→0.056).

As the HMW/LMW HA ratio decreases from 100/0 to 65/35 to 35/65 at the same HA and collagen concentrations (Formulation VI vs. Formulation VII vs. Formulation VIII), the G′ value decreases (1360→1180→932 Pa) along with the compression force (292→226→163 gmf) and extrusion force (63.6→29.2→20.0 N) whereas the G″/G′ ratio increases as the HMW/LMW ratio decreases (0.055→0.068→0.085).

As the synthesis hydration temperature is decreased from 35 to 22 to 15 to 5° C. with constant HA and collagen concentrations (Formulation XI vs. Formulation VI vs. Formulation IX vs. Formulation X), the G′ value increases (876→1360→3145→4750 Pa) whereas the hydrogel swelling decreases (257%→238%→159%→127%) along with the extrusion force (56.1˜63.6→33.8→20.1 N). The compression force is not affected by changes in hydration temperature, except at higher hydration temperatures (35° C. vs. others). Modulating the hydration temperature during the synthesis alters the solubility of the collagen and therefore, results in changes to the physical properties of the resulting hydrogels.

By reducing the salt/buffer concentration by two-thirds, a similar G′ value (5470 Pa vs. 4750 Pa), swelling (108% vs. 127%), and extrusion force (20.5 N vs. 20.1 N) is obtained for a formulation hydrated at 22° C. with a reduced salt/buffer concentration and one hydrated at 5° C. with the full salt/buffer concentration (Formulation XIII vs. Formulation X). Additionally, the synthesis using reduced salt/buffer concentration is not as sensitive to hydration temperatures of 22° C. vs. 5° C. as the synthesis using the full salt/buffer concentration. For formulations synthesized using the reduced salt/buffer concentration with hydration temperatures of 22° C. and 5° C. (Formulation XIII vs Formulation XII), the G′, swelling, and extrusion force values do not differ greatly, whereas for similar formulations synthesized using the full concentration of salt/buffer, these physical properties differ significantly (Formulation VI vs. Formulation X).

The effect of added lysine is observed by comparing similar formulations synthesized at HA: collagen concentrations of 28:2 mg/mL (Formulation XVIII vs. Formulation XVI vs. Formulation XVII). An optimal lysine:HA ratio serves to maximize the crosslinking efficiency, with the exact ratio dependent on HA molecular weight, activation reagent concentrations, and synthesis conditions. For example, the lysine:HA ratio was increased (0→0.333→0.5) for a series of formulations synthesized at HA:collagen concentrations of 28:2 mg/mL (Formulation XVIII vs. Formulation XVI vs. Formulation XVII). Physical properties such as G′ and compression force were maximized for the formulation prepared with a lysine:HA ratio of 0.333, whereas swelling and G″/G′ value were minimized for that same formulation. Higher G′ and lower swelling are generally associated with more highly crosslinked hydrogels. A lysine:HA ratio between 0 and 0.5 allows for more efficient crosslinking. Crosslinking without lysine may rely on collagen to provide the amines for crosslinking and may allow for more water-labile ester crosslinks between HA chains. Crosslinking with high lysine:HA ratios may saturate the activated carboxylic acids on the HA chains and may lead to pendant lysine molecules attached to the HA chain on one side only, rather than crosslinking between chains. These scenarios with very low or high lysine:HA ratios may lead to inefficient crosslinking and sub-optimal gel properties. By selecting the optimal lysine:HA ratio for a given indication, the physical properties of the resulting hydrogel is tuned and the desired characteristics can be achieved.

### Example 6

### In Vitro Testing of Hydrogels

### In Vitro Cell Proliferation and Viability.

Viability and proliferation of fibroblast cells in close contact with HA-Collagen hydrogels was quantified using XTT assays. 100 μL of hydrogel (n=3) was layered on the bottom of a 24-well cell culture plate with a low adhesion surface coating and place in a humidified incubator at 37° C. for 30 minutes. 50,000 adult human dermal fibroblasts in 500 μL of cell culture medium were added on top of the hydrogel beds and incubated 37° C. After 48 hours of incubation, 250 μL of XTT reagent was added to each well and incubated at 37° C. for 4 hours. The plate was then spun at 300×g for 5 minutes and 200 μL of supernatant from each well was transferred to wells of 96-well filter plate with a 20 μm mesh. The filter plate containing the XTT supernatant was spun at 300×g for 5 minutes. 100 μL of filtered supernatant from each well was transferred to a clean 96-well plate (black walls, clear bottom) and the absorbance of the supernatant was read on a microplate reader (450 nm with 630 nm background correction). The data was normalized to the XTT cell viability of fibroblasts cultured on the positive control Tissue Culture Polystyrene (TCPS).

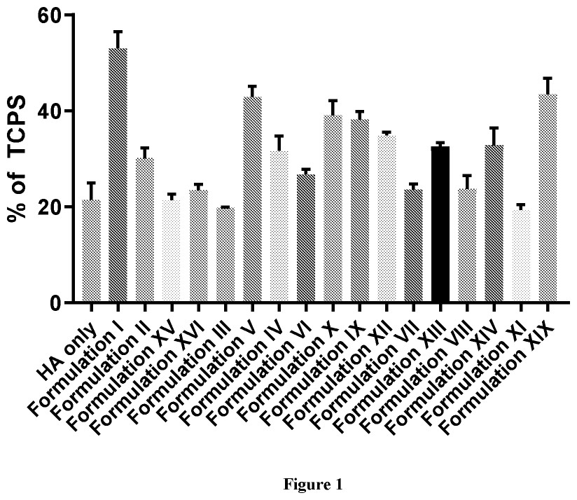

It was found that cell viability and proliferation was higher for formulations with lower HA concentrations over those similarly crosslinked formulations with the same collagen concentration and higher HA concentrations. For example, hydrogels synthesized with HMW HA and 4 mg/mL collagen but with increasing HA concentrations of 13 mg/mL (Formulation I), 20 mg/mL (Formulation II), and 25 mg/mL (Formulation III) showed proliferation values of 53%, 30%, and 20% relative to TCPS positive controls. (FIG. 1.) HA-only negative control gels showed a proliferation value of 12% relative to TCPS controls.

Additionally, the hydration temperature during the synthesis procedure shows an effect on cell viability and proliferation for similarly crosslinked formulations with the same HA and collagen concentrations. For hydrogels with HA:collagen concentrations of 24:6 mg/mL, those formulations hydrated at 5° C. (Formulation X) demonstrated higher cell proliferation than those formulations hydrated at 22° C. (Formulation VI) or 35° C. (Formulation XI) with values of 39%, 27%, and 19%, respectively. (FIG. 1.)

Cell viability and proliferation is also impacted by salt and buffer concentration during hydrogel synthesis. No change in cell response was evident when reducing the salt/buffer concentration for syntheses performed at 5° C. (Formulation X, 1 eq. vs. Formulation XII, 0.33 eq.). However, for formulations hydrated at 22° C. or 35° C., significant increases in cell viability and proliferation were observed for those formulations prepared with reduced salt/buffer concentrations (Formulation XIII, 22° C. and Formulation XIV, 35° C.) over those formulations prepared with one equivalent of salt/buffer (Formulation VI, 22° C. and Formulation XI, 35° C.). (FIG. 1.)

Formulation XIX is prepared with 20 mg/mL HA and 6 mg/mL collagen at 5° C. This formulation demonstrated higher cell viability and proliferation relative to other gels with HA concentrations at 20 mg/mL or above. (FIG. 1.)

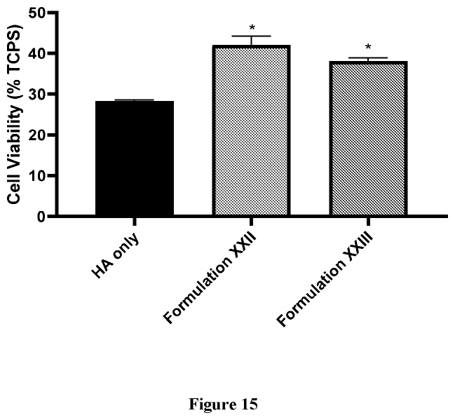

Formulations comprising a HA:Collagen ratio of 20:4 were also shown to enhanced in vitro cell response (FIG. 15, Formulation XXII).

Additionally, Formulation XIX was shown to have consistent stability and performance after autoclaving.

### In Vitro Cell Morphology.

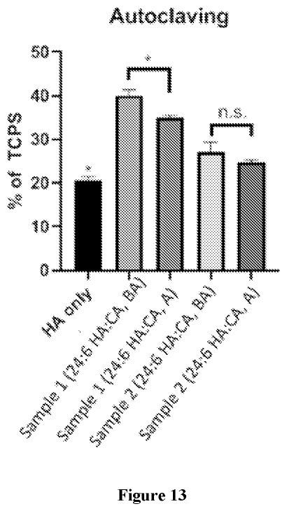

Cell morphology was analyzed to assess the effect of hydrogel formulations on cell size, shape, and cytoskeleton organization. The actin filament alignment index and morphology of fibroblast cells cultured on HA-only or HA/collagen crosslinked hydrogels was imaged and quantified. Increased actin filament alignment may correlate to the increased adhesion of cells to their substrate. Increased length to width ratios correlate to increased cell spreading on a substrate. Convex Hull to Cell Area ratio is a measurement of cell shape where a 1.0 indicates a uniformly shaped cell and values above 1 indicate a more irregularly shaped cell. Cells that are making multiple contacts with the matrix and extending/migrating exhibit a more irregular cell shape and a higher Convex Hull to Cell Area ratio value. Actin filament alignment index, length to width ratio, and convex hull to cell area ratio may be analyzed together in 3-dimensional Euclidian space. The Euclidian distance of hydrogels from a negative control, in this case non-adhesive HA-only gels, enables ranking the overall cell response to fillers. Greater Euclidian distance from HA-only controls indicates enhanced cell adhesion and spreading on the hydrogels. Hydrogels that support greater cell adhesion and spreading would be expected to induce more cell infiltration into the gel, with those cells depositing ECM within the gel matrix. Increased cell infiltration and ECM deposition could be beneficial for in vivo tissue integration into hydrogel depots. Conversely, those formulations which result in lower cell adhesion and spreading values would behave more inertly and allow for less tissue infiltration and integration. In some embodiments, the methods of making the hydrogels further comprises an autoclaving step, wherein the autoclaving does not change the properties of the hydrogels. (See FIG. 13).

In a typical procedure, hydrogels (n=3) and human dermal fibroblasts in cell culture medium were added to a 96-well cell culture plate with a low adhesion surface coating. After 48 hours of incubation, the cells were fixed in formalin and stained with Hoechst, WGA-488, and Alexa Fluor-Phalloidin. The wells were imaged with a confocal microscope and actin filament alignment (phalloidin) and cell morphology (WGA-488) were analyzed using image analysis software.

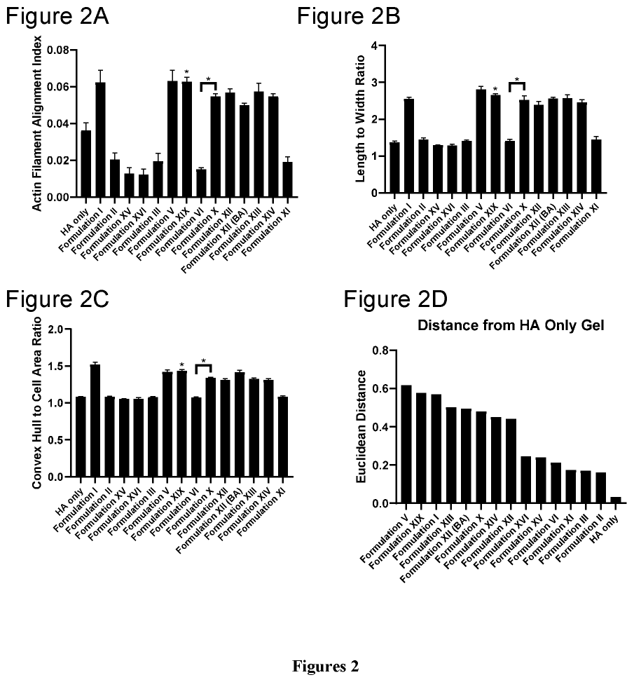

The hydration temperature during the synthesis procedure shows an effect on cell adhesion and spreading for similarly crosslinked formulations with the same HA and collagen concentrations. For hydrogels with HA:collagen concentrations of 24:6, those formulations hydrated at 5° C. (Formulation X) demonstrated increased cell adhesion (actin filament alignment index) over those formulations hydrated at 22° C. (Formulation VI) with values of 0.054 and 0.015, respectively (FIG. 2). The formulations hydrated at 5° C. (Formulation X) exhibited increased cell spreading (cell length to width ratio) over those hydrated at 22° C. (Formulation VI), with values of 2.52 and 1.40, respectively. The formulations hydrated at 5° C. (325_B) also exhibited increased convex hull to cell area ratio over those hydrated at 22° C. (Formulation VI), with values of 1.34 and 1.07, respectively. The actin filament alignment index, cell length to width ratio, and convex hull to cell area ratio of formulations hydrated at 22° C. (Formulation VI) was similar to the HA-only control. An optimized formulation (20:6 HA:Collagen, 5° C. hydration; Formulation XIX), exhibits significantly higher actin filament alignment index, length to width ratio, and convex hull to cell area ratios than HA-only gel. Ranking the hydrogels using Euclidian distance from the HA-only gel demonstrates that the optimized formulation performs higher than other HA-Collagen hydrogels.

The cell morphology analysis correlates well with the XTT cell activity assay in that Formulation X which demonstrated a higher activity than Formulation VI in the activity assay also showed evidence of enhanced cell adhesion and spreading in the morphology assay. Formulation XIX also shows higher activity than other HA-Collagen formulations and HA-only gel. Cell spreading and adhesion are linked to higher cell activity and therefore, the results of each assay are in good agreement with one another. (FIGS. 2A-2D).

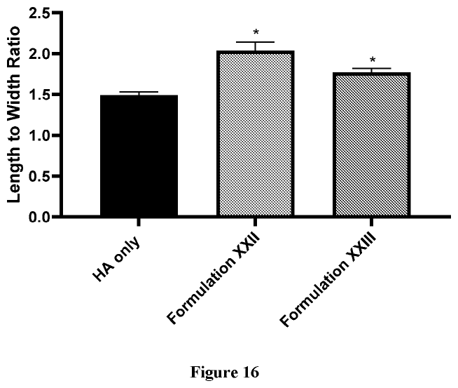

Fibroblasts cultured with Formulation XXII and Formulation XXIII exhibit significantly greater cell length to width ratio than fibroblasts cultured with HA only gel. (FIG. 16.)

### Example 7

### In Vivo Testing of Hydrogels

### Lift Capacity.

The capacity of hydrogels to support tissue projection (lift) was evaluated in vivo with a subcutaneous implantation model in rats. 125 μL of hydrogel (n=10) was injected as a subcutaneous bolus on top of the skull. A clinical 3-D imaging system (Canfield Vectra) was used to generate 3-D reconstructions of the bolus over the course of 12 weeks. The mean height of the bolus was analyzed using medical imaging software (Canfield Mirror).

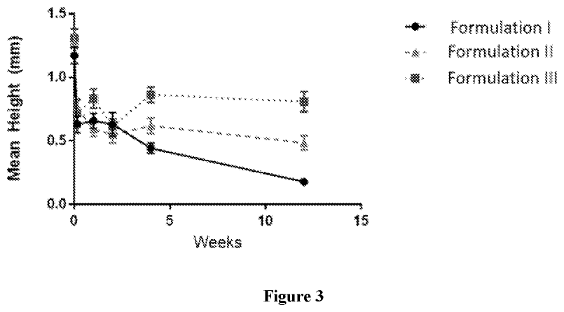

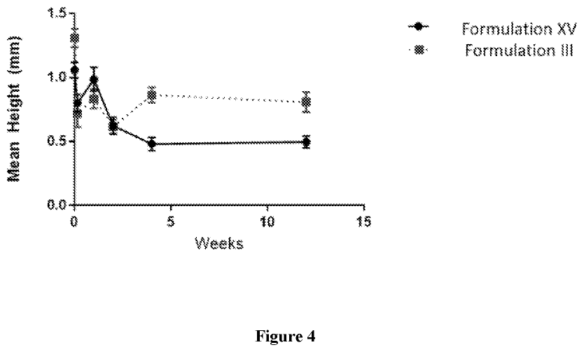

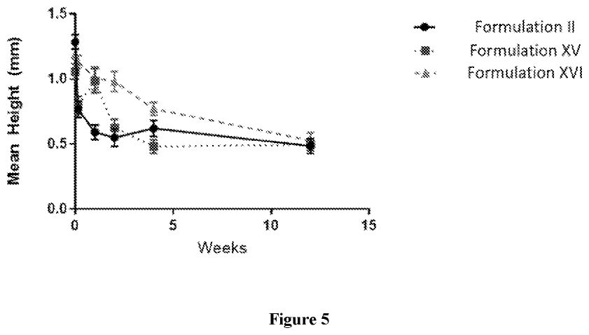

The in vivo lift capacity of a series of HA-Collagen formulations with the same collagen concentration (4 mg/mL) and HMW/LMW HA ratio (100/0) measured between 4 and 12 weeks shows a positive correlation with HA concentration. A formulation with 25 mg/mL HA (Formulation III) demonstrated more projection than formulations with 20 mg/mL (Formulation II) or 13 mg/mL (Formulation I), FIG. 3. Since the compression force increases with HA concentration for these formulations, the in vivo projection from 4-12 weeks shows a positive correlation with compression force. The in vivo lift is also dependent on the gel synthesis conditions. Two formulations contained the same HA:collagen concentrations of 25:4 mg/mL (Formulation III vs. Formulation XV), but were synthesized with different HMW/LMW HA ratios and under different crosslinking conditions and gave rise to different lift profiles from 4-12 weeks. The formulation synthesized with high MW HA at a synthesis concentration of 1× demonstrated superior lift than the formulation prepared with 10/90 HMW/LMW HA at a synthesis concentration of 1.25× (FIG. 4). Additionally, hydrogel formulations with different HA/collagen concentrations and synthesis conditions (Formulation II, Formulation XV, Formulation XVI), but with similar compression force values (166-180) showed similar in vivo lift profiles between 4 and 12 weeks (FIG. 5). Thus, by selecting the optimal composition and synthesis conditions, the desired lift profile for a given application can be obtained.

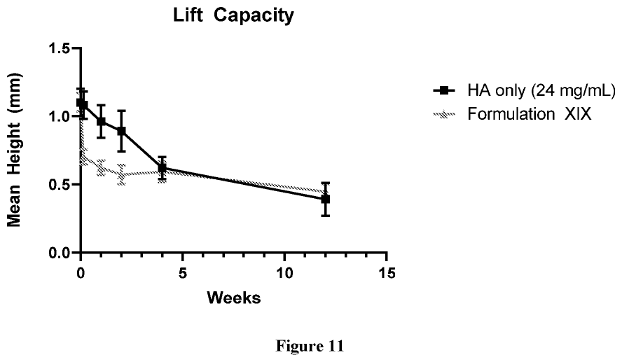

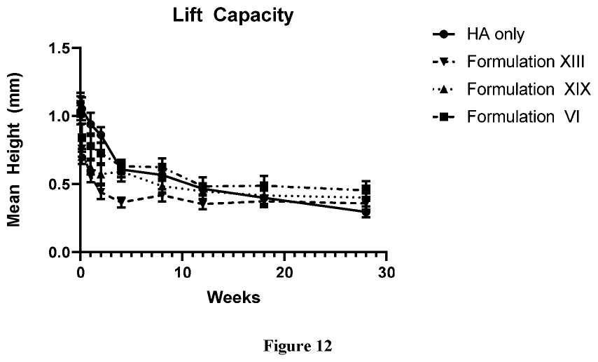

Lift capacity was also tested with Formulation XIX and a HA only formulation (FIGS. 11 and 12). As shown, Formulation XIX exhibited a similar lift capacity to a 24 mg/ml HMW HA-only gel from 4 to 28 weeks.

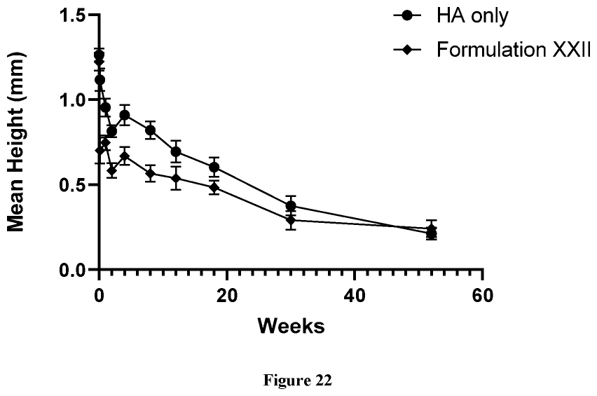

Extended (52 week) lift capacity data of Formulation XXII was tested. The lift capacity of the HA only control steadily decreased over time. In contrast, the lift capacity of the HA-Collagen gel (Formulation XXII) remains stable from 30 to 52 weeks. (FIG. 22.) This surprising result indicates that these HA-Collagen gel formulations are capable of longer lift duration than HA only gels. This correlates with better tissue ingrowth than HA only gels (see below).

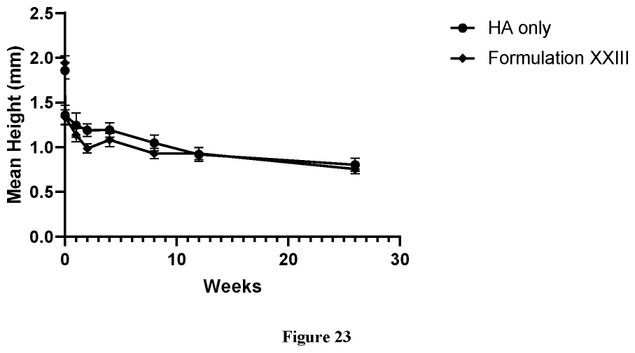

Extended (26 week) lift capacity data of Formulation XXII was tested. Formulation XXIII and the HA only comparator exhibit similar lift capacity over the course of 26 weeks. (FIG. 23.) The enhanced tissue integration of Formulation XXIII surprisingly results in enhanced duration of lift capacity and other benefits to the overall effect. For example, the newly created tissues sustain skin quality, lift capacity, and wrinkle correction.

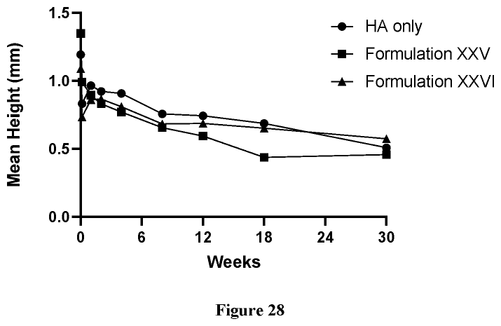

Extended (30 week) lift capacity data of Formulation XXV was tested. The lift capacity of the HA only gel steadily decreases over time. (FIG. 28.) In contrast, the lift capacity of HA-Collagen gels (Formulation XXV and XXVI) remained stable from 18 to 30 weeks. (FIG. 28.) This surprising result indicates that these HA-Collagen gel formulations are capable of longer lift duration than HA only gels. This correlates with better tissue ingrowth than HA only gels (see below).

### In Vivo Tissue Integration.

In vivo tissue integration for a range of formulations was assessed using a subcutaneous implantation model in rats. In a typical procedure, 125 μL of hydrogel was delivered as a subcutaneous bolus on the dorsal aspect of the rat. After 4 weeks the bolus was explanted, fixed in formalin, and embedded in paraffin for histology. Tissue sections were stained for hematoxylin & eosin (H&E) and colloidal iron. Immunohistochemical staining for Collagen Type I, Vimentin, CD31, and Procollagen Type I was also performed.

The tissue integration shows a negative correlation with HA concentration for formulations prepared similarly with HMW HA and 4 mg/mL collagen. The density of collagen deposited by surrounding tissue is higher for a formulation with 13 mg/mL HA (Formulation I) than for those prepared with 20 mg/mL HA (Formulation II) or 25 mg/mL (Formulation III) and is HA concentration dependent (FIGS. 6A-6E). Additionally, there are fewer areas of the injected bolus devoid of tissue for the 13 mg/mL HA formulation than for those materials with 20 mg/mL or 25 mg/mL HA. In addition to HA concentration, it would be expected that integration would be mainly dependent on the collagen concentration. However, it appears that other factors may strongly influence tissue infiltration into the bolus. For example, a formulation (Formulation XVI) with high HA concentration (28 mg/mL) and low collagen concentration (2 mg/mL) demonstrates collagen deposition throughout the bolus and very few areas devoid of tissue.

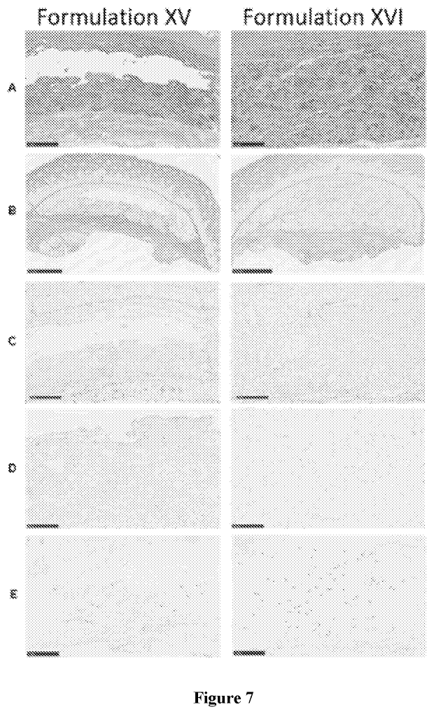

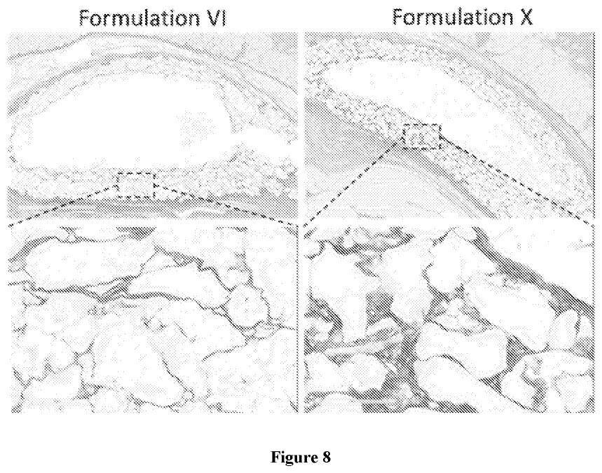

Formulation XVI was prepared with mostly LMW HA unlike the previously mentioned formulations which were prepared with HMW HA. However, the molecular weight of the HA is not the only contributing factor either, since a second formulation with an HA:collagen concentration of 25:4 mg/mL (Formulation XV) was prepared with mostly LMW HA but does not exhibit the same strong integration throughout the bolus (FIG. 7). Hydration temperature during the synthesis has been shown above to influence in vitro cell response and through subsequent studies has also been shown to affect in vivo cell infiltration and tissue integration. Two similar formulations prepared with HA:collagen concentrations of 24:6 mg/mL but with different hydration temperatures of 5° C. (Formulation X) and 22° C. (Formulation VI) show different densities of collagen deposition around the perimeter of the bolus with a higher response from the formulation prepared at 5° C. (FIG. 8). Rather than one parameter, a combination of factors including HA concentration, HA molecular weight ratio, collagen concentration, and synthesis conditions influence the extent of tissue integration. A range of tissue responses has been achieved with these materials and therefore, this tissue integration and infiltration can be tailored to a particular filler application by optimizing the aforementioned synthesis parameters.

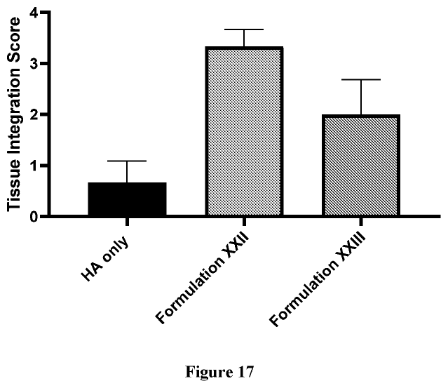

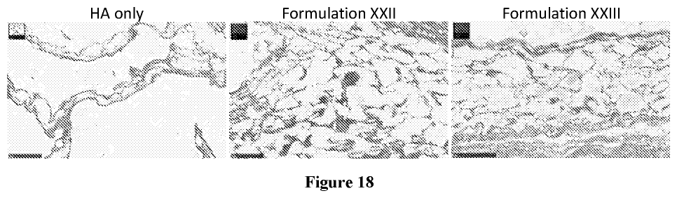

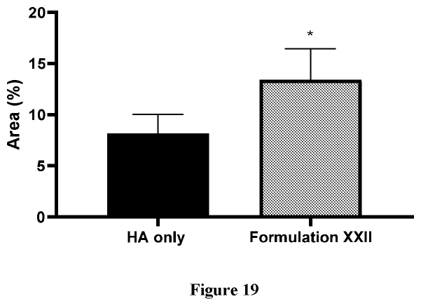

Formulation XXII and Formulation XXIII show enhanced tissue integration compared to HA only gel. (FIG. 17.) Collagen 1a staining shows fine collagen distribution around gel particles in the HA-Collagen formulations and limited deposition of Collagen 1a in the HA only gel. (FIG. 18.) Quantification of the percent positive area of Collagen 1a staining within the hydrogel bolus after 4 week subcutaneous implantation in rats demonstrates that Formulation XXII generates more Collagen 1a positive tissue that HA only hydrogel. (FIG. 19.)

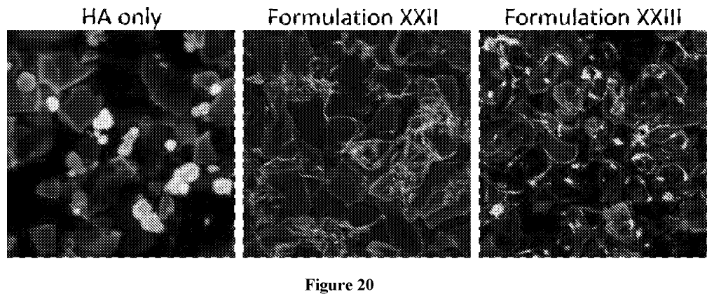

FIG. 20 shows confocal micrographs of human dermal fibroblasts cultured on HA only, Formulation XXII, or Formulation XXIII gels for 48 hours. The samples were stained for HA Binding Protein, Hoechst, and cell membrane. The created gels demonstrate marked improvements in cell adhesion when compared to HA only crosslinked products, thus demonstrating the potential of the gels to act as a scaffold for tissue integration and collagen deposition.

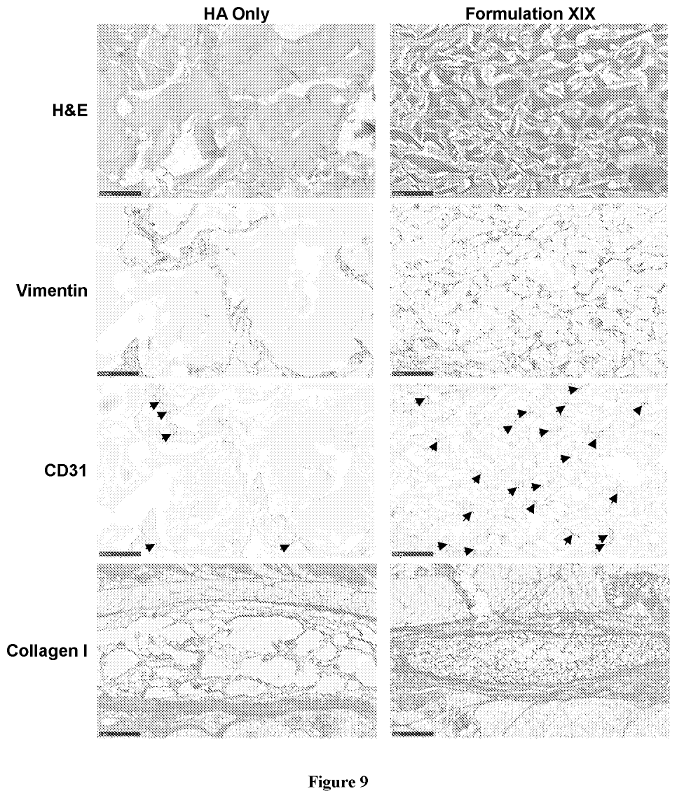

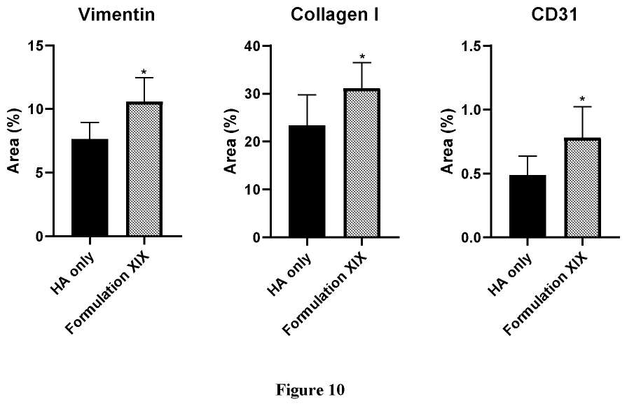

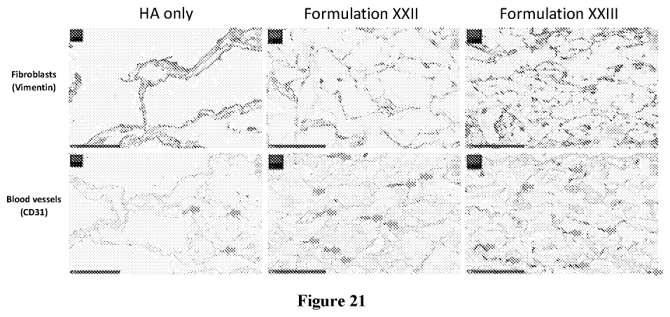

Formulation XIX was also tested for its ability to increase levels of Vimentin (fibroblasts), Collagen I and CD31. As shown, in FIGS. 9 and 10, Formulation XIX was able to increase levels of Vimentin (fibroblasts), Collagen I, and CD31 (blood vessels) in the bolus of Formulation XIX hydrogel after 12 weeks subcutaneous implantation in rats, as compared to HA only hydrogel. This corroborates with Formulation XIX having improved cell spreading and adhesion. Similarly, Formulation XXII and Formulation XXIII promote greater infiltration of fibroblasts (vimentin staining) and vascularization (CD31 staining, arrows) compared to HA only controls. (FIG. 21.) This indicates tissue regeneration in the hydrogel bolus with morphology consistent with endogenous tissues.

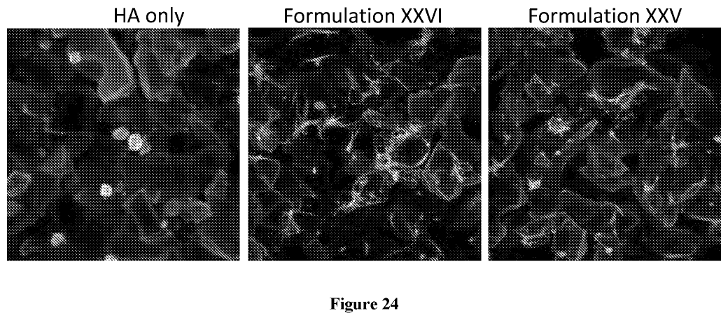

FIG. 24 shows confocal micrographs of human dermal fibroblasts cultured on HA only, Formulation XXVI, or Formulation XXV gels for 48 hours. The samples were stained for HA Binding Protein, Hoechst, and cell membrane. The Formulation XXVI and Formulation XXV gels demonstrate surprising improvements in cell adhesion when compared to HA only crosslinked products, thus demonstrating the potential of the gels to act as a scaffold for tissue integration and collagen deposition.

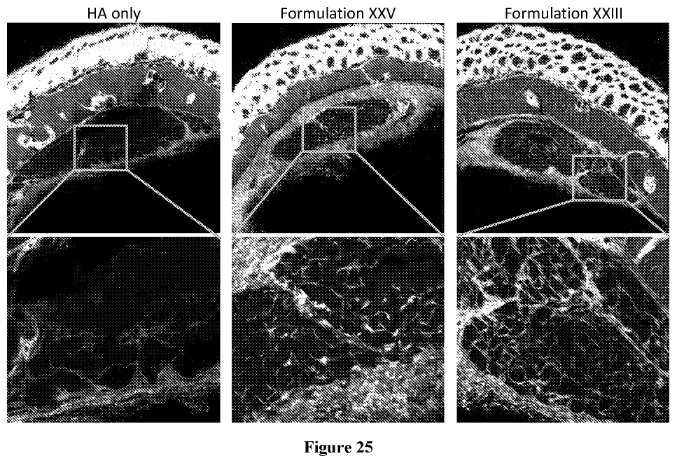

FIG. 25 shows two photon imaging of the second harmonic generation signal (white) and tissue autofluorescence (green) in rats treated with subcutaneous bolus injections of HA only, Formulation XXV, or Formulation XXIII after 12 weeks. The presence of second harmonic generation (white) indicates fully assembled fibrillar collagen formation in the HA-Collagen treated implants. Limited second harmonic generation is observed in the HA only gel.

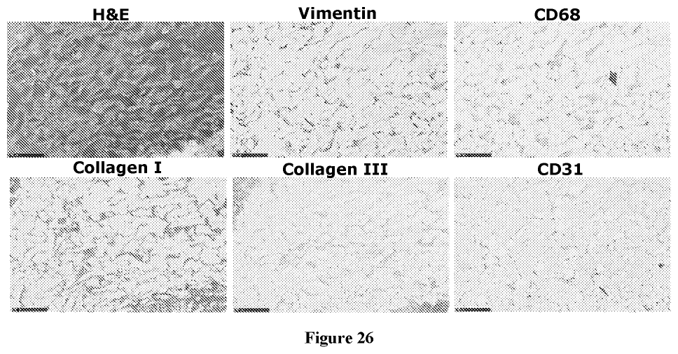

FIG. 26 shows immunohistochemistry analysis of the tissue response to Formulation XXV after 4 weeks subcutaneous implantation in rats. Formulation XXV promotes integration of tissue (H&E staining), fibroblast infiltration (vimentin), limited macrophage response (CD68), deposition of Collagen I, limited Collagen III and vascularization (CD31). This may indicate natural tissue regeneration in the hydrogel bolus. Furthermore, Formulation XXV resulted in a higher tissue integration pathology score (4.67) versus an HA only control (0.67).

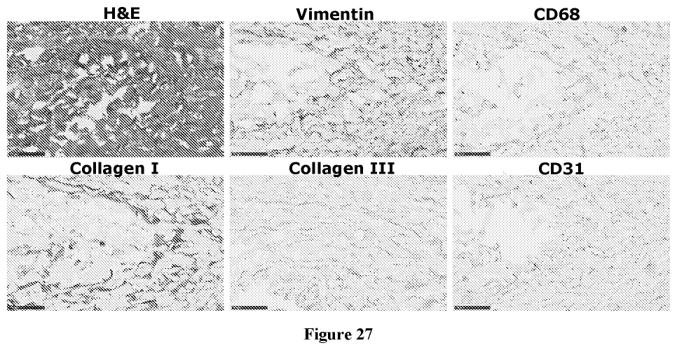

FIG. 27 shows immunohistochemistry analysis of the tissue response to Formulation XXVI after 4 weeks subcutaneous implantation in rats. Formulation XXVI promotes integration of tissue (H&E staining), fibroblast infiltration (vimentin), limited macrophage response (CD68), deposition of Collagen I, limited Collagen III and vascularization (CD31). This may indicate natural tissue regeneration in the hydrogel bolus. Furthermore, Formulation XXVI resulted in a higher tissue integration pathology score (4.17) versus an HA only control (0.67).

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A crosslinked macromolecular matrix comprising: lysine; hyaluronic acid; and collagen; wherein the hyaluronic acid is crosslinked to the collagen by at least one endogenous amine group on the collagen and/or by at least one amine group present on the lysine.

Clause 2. The crosslinked macromolecular matrix of any of the above or below Clauses, wherein the crosslinked macromolecular matrix further comprises lidocaine.

Clause 3. The crosslinked macromolecular matrix of any of the above or below Clauses, wherein the lidocaine is at a concentration in between a range of about 0.15% (w/w) to about 0.45% (w/w) in the matrix.

Clause 4. The crosslinked macromolecular matrix of any of the above or below Clauses, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.37% (w/w), about 0.39% (w/w), about 0.41% (w/w), about 0.43% (w/w), or about 0.45% (w/w) of the matrix or any concentration in between a range defined by any two aforementioned values.

Clause 5. The crosslinked macromolecular matrix of any of the above or below Clauses, wherein the lidocaine is at a concentration in between a range of about 0.27% (w/w) to about 0.33% (w/w) in the matrix.

Clause 6. The crosslinked macromolecular matrix of any of the above or below Clauses, wherein the matrix further comprises un-crosslinked HA.

Clause 7. The crosslinked macromolecular matrix of any of the above or below Clauses, wherein the un-crosslinked HA comprises a concentration of up to about 5% (w/w) within the matrix.

Clause 8. The crosslinked macromolecular matrix of any of the above or below Clauses, wherein the un-crosslinked HA comprises a concentration of about 0% (w/w), about 1% (w/w), about 2% (w/w), about 3% (w/w), about 4% (w/w), or about 5% (w/w) in the matrix, or any concentration in between a range defined by any two aforementioned values.

Clause 9. The crosslinked macromolecular matrix of any of the above or below Clauses, wherein the un-crosslinked HA comprises a concentration of about 1% (w/w) in the matrix.

Clause 10. The crosslinked macromolecular matrix of any of the above or below Clauses, wherein the un-crosslinked HA comprises a concentration of about 2% (w/w) in the matrix.

Clause 11. The crosslinked macromolecular matrix of any of the above or below Clauses, wherein the un-crosslinked HA comprises a concentration of about 5% (w/w) in the matrix.

Clause 12. The crosslinked macromolecular matrix of any of the above or below Clauses, wherein the un-crosslinked HA, improves the extrudability of the macromolecular matrix.

Clause 13. The crosslinked macromolecular matrix of any of the above or below Clauses, wherein the crosslinked macromolecular matrix is stable for about 6 months, about 12 months, about 18 months, about 24 months, about 30 months, or about 36 months or any amount of time in between a range defined by any two aforementioned values.

Clause 14. The crosslinked macromolecular matrix of any of the above or below Clauses, wherein the crosslinked macromolecular matrix is stable at a temperature in between about 4° C. and about 25° C.

Clause 15. The crosslinked macromolecular matrix of any of the above or below Clauses, wherein the crosslinked macromolecular matrix is stable at about 4° C.

Clause 16. The crosslinked macromolecular matrix of any of the above or below Clauses, wherein the crosslinked macromolecular matrix is stable at about 25° C.

Clause 17. The crosslinked macromolecular matrix of any of the above or below Clauses, wherein the crosslinked macromolecular matrix is stable about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36 months, or any time in between a range defined by any two aforementioned values.

Clause 18. The crosslinked macromolecular matrix of any of the above or below Clauses, wherein the crosslinked macromolecular matrix has minimal degradation at about 6 months, about 12 months, about 18 months, about 24 months, about 30 months, or about 36 months or any amount of time in between a range defined by any two aforementioned values.

Clause 19. The crosslinked macromolecular matrix of any of the above or below Clauses, wherein the matrix comprises an elastic modulus (G′) of about 30 Pa to about 10,000 Pa, or any elastic modulus in between a range defined by any two aforementioned values.

Clause 20. The crosslinked macromolecular matrix of any of the above or below Clauses, wherein the matrix comprises an elastic modulus (G′) of about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, about 3000 Pa, about 3100 Pa, about 3200 Pa, about 3300 Pa, about 3400 Pa, about 3500 Pa, about 3600 Pa, about 3700 Pa, about 3800 Pa, about 3900 Pa, about 4000 Pa, about 4100 Pa, about 4200 Pa, about 4300 Pa, about 4400 Pa, about 4500 Pa, about 4600 Pa, about 4700 Pa, about 4800 Pa, about 4900 Pa, about 5000 Pa, about 5100 Pa, about 5200 Pa, about 5300 Pa, about 5400 Pa, about 5500 Pa, about 5600 Pa, about 5700 Pa, about 5800 Pa, about 5900 Pa, about 6000 Pa, about 6100 Pa, about 6200 Pa, about 6300 Pa, about 6400 Pa, about 6500 Pa, about 6600 Pa, about 6700 Pa, about 6800 Pa, about 6900 Pa, about 7000 Pa, about 7100 Pa, about 7200 Pa, about 7300 Pa, about 7400 Pa, about 7500 Pa, about 7600 Pa, about 7700 Pa, about 7800 Pa, about 7900 Pa, about 8000 Pa, about 8100 Pa, about 8200 Pa, about 8300 Pa, about 8400 Pa, about 8500 Pa, about 8600 Pa, about 8700 Pa, about 8800 Pa, about 8900 Pa, about 9000 Pa, about 9100 Pa, about 9200 Pa, about 9300 Pa, about 9400 Pa, about 9500 Pa, about 9600 Pa, about 9700 Pa, about 9800 Pa, about 9900 Pa, or about 10,000 Pa or any elastic modulus in between a range defined by any two aforementioned values.

Clause 21. The crosslinked macromolecular matrix of any of the above or below Clauses, wherein the matrix comprises a compression force value of about 10 gmf, about 20 gmf, about 30 gmf, about 40 gmf, about 50 gmf, about 60 gmf, about 70 gmf, about 80 gmf, about 90 gmf, about 100 gmf, about 110 gmf, about 120 gmf, about 130 gmf, about 140 gmf, about 150 gmf, about 160 gmf, about 170 gmf, about 180 gmf, about 190 gmf, about 200 gmf, about 210 gmf, about 220 gmf, about 230 gmf, about 240 gmf, about 250 gmf, about 260 gmf, about 270 gmf, about 280 gmf, about 290 gmf, about 300 gmf, about 310 gmf, about 320 gmf, about 330 gmf, about 340 gmf, about 350 gmf, about 360 gmf, about 370 gmf, about 380 gmf, about 390 gmf, about 400 gmf, about 410 gmf, about 420 gmf, about 430 gmf, about 440 gmf, about 450 gmf, about 460 gmf, about 470 gmf, about 480 gmf, about 490 gmf, about 500 gmf, about 510 gmf, about 520 gmf, about 530 gmf, about 540 gmf, about 550 gmf, about 560 gmf, about 570 gmf, about 580 gmf, about 590 gmf or about 600 gmf or any compression force value in between a range defined by any two aforementioned values.

Clause 22. The crosslinked macromolecular matrix of any of the above or below Clauses, wherein the matrix comprises a compression force value of about 100 gmf, about 200 gmf, about 300 gmf, about 400 gmf, about 500 gmf or about 600 gmf or any compression force value in between a range defined by any two aforementioned values.

Clause 23. The crosslinked macromolecular matrix of any of the above or below Clauses, wherein the hyaluronic acid is at a concentration of about 5 mg/ml, about 6 mg/ml, about 8 mg/ml, about 10 mg/ml, about 12 mg/ml, about 14 mg/ml, about 16 mg/ml, about 18 mg/ml, about 20 mg/ml, about 22 mg/ml, about 24 mg/ml, about 26 mg/ml, about 28 mg/ml, about 30 mg/ml, about 32 mg/ml, about 34 mg/ml or about 36 mg/ml or any concentration in between a range defined by any two aforementioned values.

Clause 24. The crosslinked macromolecular matrix of any of the above or below Clauses, wherein the collagen comprises Type I collagen.

Clause 25. The crosslinked macromolecular matrix of any of the above or below Clauses, wherein the collagen comprises Type II collagen.

Clause 26. The crosslinked macromolecular matrix of any of the above or below Clauses, wherein the collagen comprises Type III collagen.

Clause 27. The crosslinked macromolecular matrix of any of the above or below Clauses, wherein the collagen comprises 0% to 3% Type II collagen.

Clause 28. The crosslinked macromolecular matrix of any of the above or below Clauses, wherein the collagen comprises 1%-3% Type I collagen.

Clause 29. The crosslinked macromolecular matrix of any of the above or below Clauses, wherein the matrix comprises about 0% to about 3% type III collagen.

Clause 30. The crosslinked macromolecular matrix of any of the above or below Clauses, wherein the collagen comprises about 97% to about 99% Type I collagen.

Clause 31. The crosslinked macromolecular matrix of any of the above or below Clauses, wherein the collagen comprises a mixture of both Type I and Type III collagen.

Clause 32. The crosslinked macromolecular matrix of any of the above or below Clauses, wherein the collagen comprises a concentration of about 1 mg/ml, about 2 mg/ml, about 4 mg/ml, about 6 mg/ml, about 8 mg/ml, about 10 mg/ml, about 12 mg/ml, about 14 mg/ml or any concentration in between a range defined by any two aforementioned values.

Clause 33. The crosslinked macromolecular matrix of any of the above or below Clauses, wherein the crosslinked macromolecular matrix further comprising a salt.

Clause 34. The crosslinked macromolecular matrix of any of the above or below Clauses, wherein the crosslinked macromolecular matrix comprises NaCl in a range between about 50 mM to about 400 mM.

Clause 35. The crosslinked macromolecular matrix of any of the above or below Clauses, wherein the crosslinked macromolecular matrix comprises NaCl, wherein the NaCl comprises a concentration of about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, or about 400 mM, or any concentration in between a range defined by any two aforementioned values.

Clause 36. The crosslinked macromolecular matrix of any of the above or below Clauses, wherein the crosslinked macromolecular matrix comprises NaCl, wherein the NaCl comprises a concentration of about 150 mM.

Clause 37. The crosslinked macromolecular matrix of any of the above or below Clauses, wherein the crosslinked macromolecular matrix comprises phosphate buffer of about 0.01M, NaCl of about 137 mM and KCl in a concentration of about 2.7 mM.

Clause 38. The crosslinked macromolecular matrix of any of the above or below Clauses, wherein the crosslinked macromolecular matrix is formulated for injection or use with a needle and/or cannula.

Clause 39. The crosslinked macromolecular matrix of any of the above or below Clauses, wherein the hyaluronic acid component has an average molecular weight of about 20,000 Daltons to about 10,000,000 Daltons.

Clause 40. The crosslinked macromolecular matrix of any of the above or below Clauses, wherein the hyaluronic acid component has an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

Clause 41. The crosslinked macromolecular matrix of any of the above or below Clauses, wherein the hyaluronic acid comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

Clause 42. A composition comprising: hyaluronic acid; collagen; lysine; and a buffer; and wherein the composition is an aqueous hydrogel.

Clause 43. The composition of any of the above or below Clauses, wherein the hyaluronic acid is crosslinked to the collagen by at least one endogenous amine group on the collagen and/or by at least one amine group present on the lysine.

Clause 44. The composition of any of the above or below Clauses, wherein the composition further comprises lidocaine.

Clause 45. The composition of any of the above or below Clauses, wherein the lidocaine is at a concentration in between a range of about 0.15% (w/w) to about 0.45% (w/w) in the matrix.

Clause 46. The composition of any of the above or below Clauses, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.37% (w/w), about 0.39% (w/w), about 0.41% (w/w), about 0.43% (w/w), or about 0.45% (w/w) of the composition or any concentration in between a range defined by any two aforementioned values.

Clause 47. The composition of any of the above or below Clauses, wherein the composition further comprises un-crosslinked HA.

Clause 48. The composition of any of the above or below Clauses, wherein the un-crosslinked-crosslinked HA comprises a concentration of up to about 5% (w/w) within the composition.

Clause 49. The composition of any of the above or below Clauses, wherein the un-crosslinked HA comprises a concentration of about 0% (w/w), about 1% (w/w), about 2% (w/w), about 3% (w/w), about 4% (w/w), about 5% (w/w) in the composition or any concentration in between a range defined by any two aforementioned values.

Clause 50. The composition of any of the above or below Clauses, wherein the un-crosslinked HA comprises a concentration of about 1% (w/w) in the composition.

Clause 51. The composition of any of the above or below Clauses, wherein the un-crosslinked HA comprises a concentration of about 2% (w/w) in the composition.

Clause 52. The composition of any of the above or below Clauses, wherein the un-crosslinked HA comprises a concentration of about 5% (w/w) in the composition.

Clause 53. The composition of any of the above or below Clauses, wherein the un-crosslinked HA, improves the extrudability of the composition.

Clause 54. The composition of any of the above or below Clauses, wherein the buffer is phosphate buffered saline.

Clause 55. The composition of any of the above or below Clauses, wherein the hyaluronic acid comprises an average molecular weight of about 20,000 Daltons to about 10,000,000 Daltons.

Clause 56. The composition of any of the above or below Clauses, wherein the hyaluronic acid comprises a mixture of hyaluronic acid components with different molecular weights, wherein the mixture comprises hyaluronic acid with a molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

Clause 57. The composition of any of the above or below Clauses, wherein the collagen comprises collagen type I.

Clause 58. The composition of any of the above or below Clauses, wherein the collagen comprises collagen type II.

Clause 59. The composition of any of the above or below Clauses, wherein the collagen comprises collagen type III.

Clause 60. The composition of any of the above or below Clauses, wherein the composition comprises a viscosity of about 4,000 Pa S, about 4100 Pa S, about 4200 Pa S, about 4300 Pa S, about 4400 Pa S, about 4500 Pa S, about 4600 Pa S, about 4700 Pa S, about 4800 Pa S, about 4900 Pa S, about 5000 Pa S, about 5100 Pa S, about 5200 Pa S, about 5300 Pa S, about 5400 Pa S, about 5500 Pa S, about 5600 Pa S, about 5700 Pa S, about 5800 Pa S, about 5900 Pa S, about 6000 Pa S, about 6100 Pa S, about 6200 Pa S, about 6300 Pa S, about 6400 Pa S, about 6500 Pa S, about 6600 Pa S, about 6700 Pa S, about 6800 Pa S, about 6900 Pa S, about 7000 Pa S, about 7100 Pa S, about 7200 Pa S, about 7300 Pa S, about 7400 Pa S, about 7500 Pa S, about 7600 Pa S, about 7700 Pa S, about 7800 Pa S, about 7900 Pa S, about 8000 Pa S, about 8100 Pa S, about 8200 Pa S, about 8300 Pa S, about 8400 Pa S, about 8500 Pa S, about 8600 Pa S, about 8700 Pa S, about 8800 Pa S, about 8900 Pa S, about 9000 Pa S, about 9100 Pa, about 9200 Pa S, about 9300 Pa S, about 9400 Pa S, about 9500 Pa S, about 9600 Pa S, about 9700 Pa S, about 9800 Pa S, about 9900 Pa S, or about 10,000 Pa S or any viscosity in between a range defined by any two aforementioned values.

Clause 61. The composition of any of the above or below Clauses, wherein the composition comprises a tan delta parameter (G″/G′) of about 0.01 to about 0.5.

Clause 62. The composition of any of the above or below Clauses, wherein the composition comprises a tan delta parameter (G″/G′) of about 0.01, about 0.05, about 0.10, about 0.15, about 0.20, about 0.25, about 0.30, about 0.35, about 0.40, about 0.45 or about 0.50 or any tan delta parameter in between a range defined by any two aforementioned values.

Clause 63. The composition of any of the above or below Clauses, wherein the composition is stable for about 6 months, about 12 months, about 18 months, about 24 months, about 30 months, or about 36 months, or any amount of time in between a range defined by any two aforementioned values.

Clause 64. The composition of any of the above or below Clauses, wherein the composition is stable at about 4° C.

Clause 65. The composition of any of the above or below Clauses, wherein the composition is stable at about 25° C.

Clause 66. The composition of any of the above or below Clauses, wherein the composition has minimal degradation at about 6 months, about 12 months, about 18 months, about 24 months, about 30 months, or about 36 months, or any amount of time in between a range defined by any two aforementioned values.

Clause 67. A method of crosslinking hyaluronic acid and collagen comprising: dissolving collagen, hyaluronic acid and lysine in an aqueous solution to form an aqueous pre-reaction solution, wherein the aqueous pre-reaction solution comprises a pH between about 4 and about 6; and preparing a second solution comprising: a water soluble carbodiimide; and an N-hydroxysuccinimide or an N-hydroxysulfosuccinimide; and adding the second solution to the aqueous pre-reaction solution to form a crosslinking reaction mixture; and reacting the crosslinking reaction mixture by crosslinking the hyaluronic acid and the collagen with lysine; wherein the hyaluronic acid is crosslinked to the collagen by at least one endogenous amine group on the collagen and/or by at least one amine group present on the lysine; and wherein the HA and collagen undergo minimal degradation and the structure of the HA and collagen remains intact, thereby forming a crosslinked macromolecular matrix.

Clause 68. The method of any of the above or below Clauses, wherein the aqueous pre-reaction solution comprises a pH of about 4.0, about 4.5, about 5.0, about 5.5 or about 6.0, or any pH in between a range defined by any two aforementioned values.

Clause 69. The method of any of the above or below Clauses, wherein the method further comprises adding lidocaine to the crosslinked macromolecular matrix.

Clause 70. The method of any of the above or below Clauses, wherein the lidocaine is added to a concentration in between a range of about 0.15% (w/w) to about 0.45% (w/w) within the crosslinked macromolecular matrix.

Clause 71. The method of any of the above or below Clauses, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.37% (w/w), about 0.39% (w/w), about 0.41% (w/w), about 0.43% (w/w), or about 0.45% (w/w) of the matrix, or any concentration in between a range defined by any two aforementioned values.

Clause 72. The method of any of the above or below Clauses, wherein the method further comprises providing an activating agent comprising a triazole, a fluorinated phenol, a succinimide, or a sulfosuccinimide.

Clause 73. The method of any of the above or below Clauses, wherein the method is performed at a temperature of about 2° C., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., about 34° C., or about 36° C. or a temperature in between a range defined by any two aforementioned values.

Clause 74. The method of any of the above or below Clauses, wherein the reacting step is performed between about 4 and about 35° C.

Clause 75. The method of any of the above or below Clauses, wherein the reacting step is performed at about 4° C. or about 22° C.

Clause 76. The method of any of the above or below Clauses, wherein the method further comprises purifying the crosslinked macromolecular matrix, wherein the purifying step is performed using dialysis.

Clause 77. The method of any of the above or below Clauses, wherein the purifying step is performed between 2° C.-30° C.

Clause 78. The method of any of the above or below Clauses, wherein the dialysis is performed at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., or any temperature in between a range defined by any two aforementioned values.

Clause 79. The method of any of the above or below Clauses, wherein the purifying step is performed at about 2° C. to about 8° C.

Clause 80. The method of any of the above or below Clauses, wherein the crosslinking reaction is performed at about 2° C. to about 35° C.

Clause 81. The method of any of the above or below Clauses, wherein the crosslinking reaction is performed at about 2° C. to about 8° C.

Clause 82. The method of any of the above or below Clauses, wherein the method is performed below room temperature.

Clause 83. The method of any of the above or below Clauses, wherein the pH of the crosslinking reaction mixture is between about 4.0 to about 6.0.

Clause 84. The method of any of the above or below Clauses, wherein the pre-reaction solution comprises a salt, wherein the salt comprises sodium chloride at a concentration of about 50 mM, about 75 mM, about 100 mM, about 125 mM, about 150 mM, about 175 mM, about 200 mM, about 225 mM, about 250 mM, about 275 mM, about 300 mM, 325 mM, about 350 mM, about 375 mM, or about 400 mM, or any concentration in between a ranged defined by any two aforementioned values, in the crosslinking reaction mixture.

Clause 85. The method of any of the above or below Clauses, wherein the water soluble carbodiimide is 1-ethyl-3-(3-dimethylaminopropyl)carbodiimide at a concentration of about 20 mM to about 200 mM in the crosslinking reaction mixture.

Clause 86. The method of any of the above or below Clauses, wherein the water soluble carbodiimide is 1-ethyl-3-(3-dimethylaminopropyl)carbodiimide at a concentration of about 20 mM, about 40 mM, about 60 mM, about 80 mM, about 100 mM, about 120 mM, about 140 mM, about 160 mM, about 180 mM or about 200 mM, or any concentration in between a range defined by any to aforementioned values.

Clause 87. The method of any of the above or below Clauses, wherein the water soluble carbodiimide and hyaluronic acid is at a mole to mole ratio of water soluble carbodiimide: hyaluronic acid repeat unit between about 0.5 to about 2.0.

Clause 88. The method of any of the above or below Clauses, wherein the water soluble carbodiimide and hyaluronic acid is at a mole to mole ratio of water soluble carbodiimide: hyaluronic acid repeat unit of about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0.

Clause 89. The method of any of the above or below Clauses, wherein the lysine and hyaluronic acid are at a mole:mole (lysine:HA repeat unit) ratio between about 0.01 to about 0.6.

Clause 90. The method of any of the above or below Clauses, wherein the lysine and hyaluronic acid are at a mole:mole (lysine:HA repeat unit) ratio of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59 or about 0.6.

Clause 91. The method of any of the above or below Clauses, wherein the method further comprises adding un-crosslinked HA to the crosslinked macromolecular matrix.

Clause 92. The method of any of the above or below Clauses, wherein the un-crosslinked HA is added to a concentration of up to 5% w/w within the crosslinked macromolecular matrix.

Clause 93. The method of any of the above or below Clauses, wherein the un-crosslinked HA is added to a concentration of about 0% (w/w), about 1% (w/w), about 2% (w/w), about 3% (w/w), about 4% (w/w), or about 5% (w/w) in the matrix, or any concentration in between a range defined by any two aforementioned values.

Clause 94. The method of any of the above or below Clauses, wherein the un-crosslinked HA added to a concentration of about 1% (w/w) in the matrix.

Clause 95. The method of any of the above or below Clauses, wherein the un-crosslinked HA is added to a concentration of about 3% (w/w) in the matrix.

Clause 96. The method of any of the above or below Clauses, wherein the un-crosslinked HA is added to a concentration of about 5% (w/w) in the matrix.

Clause 97. The method of any of the above or below Clauses, wherein the method further comprises sterilizing the crosslinked macromolecular matrix, the method comprising: transferring the crosslinked macromolecular matrix into a container, for steam sterilization; and sterilizing the hydrogel by steam sterilization.

Clause 98. The method of any of the above or below Clauses, wherein the container is a syringe.

Clause 99. The method of any of the above or below Clauses, wherein the method further comprises dialyzing the crosslinked macromolecular matrix, wherein the dialysis is through a membrane having a molecular weight cutoff of about 1000 Daltons to about 100,000 Daltons, and wherein the dialyzing is performed prior to sterilization.

Clause 100. The method of any of the above or below Clauses, wherein the dialysis is performed in phosphate buffered saline.

Clause 101. The method of any of the above or below Clauses, wherein the hyaluronic acid in the pre-reaction solution hydrates for at least about 60 minutes prior to adding the second solution.

Clause 102. The method of any of the above or below Clauses, wherein the crosslinking reaction mixture is performed for about 16 hours to about 24 hours.

Clause 103. A crosslinked macromolecular matrix prepared by a process of any of the above or below methods.

Clause 104. A method of improving an aesthetic quality of an anatomic feature of a human being, the method comprising: injecting a composition into a tissue of the human being to thereby improve the aesthetic quality of the anatomic feature; wherein the composition comprises a crosslinked macromolecular matrix comprising: hyaluronic acid; lysine; and collagen; wherein the hyaluronic acid is crosslinked to the collagen by at least one endogenous amine group on the collagen and/or by at least one amine group present on the lysine.

Clause 105. The method of any of the above or below Clauses, wherein the crosslinked macromolecular matrix further comprises lidocaine.

Clause 106. The method of any of the above or below Clauses, wherein the crosslinked macromolecular matrix further comprises un-crosslinked HA.

Clause 107. The method of any of the above or below Clauses, wherein the hyaluronic acid component has an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

Clause 108. The method of any of the above or below Clauses, wherein the hyaluronic acid comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 1,000,000 Daltons and/or any hyaluronic acid with a molecular weight within a range in between any two aforementioned values.

Clause 109. The method of any of the above or below Clauses, wherein the collagen comprises collagen type I and/or collagen type III.

Clause 110. A method of improving the appearance of an individual, the method comprising: injecting a composition into a tissue of the individual at an injection site to thereby improve the aesthetic quality of an anatomic feature, wherein infiltrating cells from the tissue integrate into the composition within the injection site, depositing new collagen within the composition; wherein the composition comprises a crosslinked macromolecular matrix comprising: hyaluronic acid; lysine; and collagen; wherein the hyaluronic acid is crosslinked to the collagen by at least one endogenous amine group on the collagen and/or by at least one amine group present on the lysine; and wherein the tissue injected by the composition is shown to have tissue integration and collagen deposition and blood vessel formation.

Clause 111. The method of any of the above or below Clauses, wherein the composition further comprises lidocaine.

Clause 112. The method of any of the above or below Clauses, wherein the composition further comprises un-crosslinked HA.

Clause 113. The method of any of the above or below Clauses, wherein the composition is injected into a chin, jaw line, lips, or nasolabial fold.

Clause 114. The method of any of the above or below Clauses, wherein the method improves symmetry among facial features.

Clause 115. The method of any of the above or below Clauses, wherein the method enhances and restores volume to facial features.

Clause 116. The method of any of the above or below Clauses, wherein the method augments, corrects, restores or creates volume in the chin, lips, jaw line, or nasolabial fold.

Clause 117. The method of any of the above or below Clauses, wherein the composition is injected into tear troughs of the individual.

Clause 118. The method of any of the above or below Clauses, wherein the composition is injected into an area comprising dermal atrophy and/or fat pad atrophy.

Clause 119. The method of any of the above or below Clauses, wherein the method provides a natural look, feel and movement in the tissue receiving the injection, wherein the composition leads to increased infiltration of collagen from tissue surrounding the injection site.

Clause 120. The method of any of the above or below Clauses, wherein there is an enhanced duration of the composition as a result of tissue integration into the injection site.

Clause 121. The method of any of the above or below Clauses, wherein the method improves hydration and elasticity of skin surrounding the injection site.

Clause 122. A method of increasing infiltration of collagen into a tissue, the method comprising: injecting a composition into the tissue of an individual, thereby creating a dermal filler depot comprising the composition, wherein the composition comprises a crosslinked macromolecular matrix comprising: hyaluronic acid; lysine; and collagen; wherein the hyaluronic acid is crosslinked to the collagen by at least one endogenous amine group on the collagen and/or by at least one amine group present on the lysine; and wherein cells from the tissue surrounding the dermal filler depot infiltrates the dermal filler depot comprising the composition, wherein the cells integrate into the composition and deposit new collagen into the composition, thereby creating infiltrated tissue within the composition and wherein blood vessels connect the infiltrated tissue within the composition to a blood supply of the individual's body.

Clause 123. The method of any of the above or below Clauses, wherein the matrix further includes lidocaine.

Clause 124. The method of any of the above or below Clauses, wherein the composition further comprises un-crosslinked HA.

Clause 125. The method of any of the above or below Clauses, wherein the hyaluronic acid comprises an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any other molecular weight in between a range defined by any two aforementioned values.

Clause 126. The method of any of the above or below Clauses, wherein the hyaluronic acid comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

Clause 127. The method of any of the above or below Clauses, wherein the collagen comprises collagen type I, collagen type II and/or collagen type III.

Clause 128. The method of any of the above or below Clauses, wherein the composition comprises about 13 mg/ml hyaluronic acid.

Clause 129. The method of any of the above or below Clauses, wherein the composition comprises about 20 mg/ml hyaluronic acid, about 22 mg/ml hyaluronic acid, about 24 mg/ml, about 26 mg/ml hyaluronic acid, about 28 mg/ml hyaluronic acid or about 30 mg/ml hyaluronic acid.

Clause 130. The method of any of the above or below Clauses, wherein the product is injected into the superficial dermis to improve skin quality, fine lines, or roughness.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The terms “a,” “an,” “the” and similar referents used in the context of describing the inventions (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the inventions and does not pose a limitation on the scope of the inventions otherwise claimed. No language in the specification should be construed as indicating any non-claimed element essential to the practice of the inventions.

Groupings of alternative elements or embodiments of the inventions disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this inventions are described herein, including the best mode known to the inventors for carrying out the inventions. Of course, variations on these described embodiments will become apparent to those of ordinary skill in the art upon reading the foregoing description. The inventor expects skilled artisans to employ such variations as appropriate, and the inventors intend for the inventions to be practiced otherwise than specifically described herein. Accordingly, the inventions include all modifications and equivalents of the subject matter recited in the claims appended hereto as permitted by applicable law. Moreover, any combination of the above-described elements in all possible variations thereof is encompassed by the inventions unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the inventions so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the inventions disclosed herein are illustrative of the principles of the present inventions. Other modifications that may be employed are within the scope of the inventions. Thus, by way of example, but not of limitation, alternative configurations of the present inventions may be utilized in accordance with the teachings herein. Accordingly, the present inventions are not limited to that precisely as shown and described.

What is claimed is:

1. A crosslinked macromolecular matrix comprising:
lysine;
hyaluronic acid; and
collagen;
wherein the hyaluronic acid is crosslinked to the collagen by at least one endogenous amine group on the collagen and/or by at least one amine group present on the lysine.

2. The crosslinked macromolecular matrix of claim 1, wherein the crosslinked macromolecular matrix further comprises lidocaine.

3. The crosslinked macromolecular matrix of claim 1 or 2, wherein the lidocaine is at a concentration in between a range of about 0.15% (w/w) to about 0.45% (w/w) in the matrix.

4. The crosslinked macromolecular matrix of any one of claims 1-3, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.37% (w/w), about 0.39% (w/w), about 0.41% (w/w), about 0.43% (w/w), or about 0.45% (w/w) of the matrix or any concentration in between a range defined by any two aforementioned values.

5. The crosslinked macromolecular matrix of any one of claims 1-4, wherein the lidocaine is at a concentration in between a range of about 0.27% (w/w) to about 0.33% (w/w) in the matrix.

6. The crosslinked macromolecular matrix of any one of claims 1-Error! Reference source not found., wherein the matrix further comprises un-crosslinked HA.

7. The crosslinked macromolecular matrix of claim 6, wherein the un-crosslinked HA comprises a concentration of up to about 5% (w/w) within the matrix.

8. The crosslinked macromolecular matrix of claim 6 or 7, wherein the un-crosslinked HA comprises a concentration of about 0% (w/w), about 1% (w/w), about 2% (w/w), about 3% (w/w), about 4% (w/w), or about 5% (w/w) in the matrix, or any concentration in between a range defined by any two aforementioned values.

9. The crosslinked macromolecular matrix of any one of claims 6-8, wherein the un-crosslinked HA comprises a concentration of about 1% (w/w) in the matrix.

10. The crosslinked macromolecular matrix of any one of claims 6-8, wherein the un-crosslinked HA comprises a concentration of about 2% (w/w) in the matrix.

11. The crosslinked macromolecular matrix of any one of claims 6-8, wherein the un-crosslinked HA comprises a concentration of about 5% (w/w) in the matrix.

12. The crosslinked macromolecular matrix of any one of claims 6-11, wherein the un-crosslinked HA, improves the extrudability of the macromolecular matrix.

13. The crosslinked macromolecular matrix of any one of claims 1-12, wherein the crosslinked macromolecular matrix is stable for about 6 months, about 12 months, about 18 months, about 24 months, about 30 months, or about 36 months or any amount of time in between a range defined by any two aforementioned values.

14. The crosslinked macromolecular matrix of any one of claims 1-13, wherein the crosslinked macromolecular matrix is stable at a temperature in between about 4° C. and about 25° C.

15. The crosslinked macromolecular matrix of any one of claims 1-14, wherein the crosslinked macromolecular matrix is stable at about 4° C.

16. The crosslinked macromolecular matrix of any one of claims 1-15, wherein the crosslinked macromolecular matrix is stable at about 25° C.

17. The crosslinked macromolecular matrix of any one of claims 1-16, wherein the crosslinked macromolecular matrix is stable about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36 months, or any time in between a range defined by any two aforementioned values.

18. The crosslinked macromolecular matrix of any one of claims 1-17, wherein the crosslinked macromolecular matrix has minimal degradation at about 6 months, about 12 months, about 18 months, about 24 months, about 30 months, or about 36 months or any amount of time in between a range defined by any two aforementioned values.

19. The crosslinked macromolecular matrix of any one of claims 1-18, wherein the matrix comprises an elastic modulus (G′) of about 30 Pa to about 10,000 Pa, or any elastic modulus in between a range defined by any two aforementioned values.

20. The crosslinked macromolecular matrix of any one of claims 1-19, wherein the matrix comprises an elastic modulus (G′) of about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, about 3000 Pa, about 3100 Pa, about 3200 Pa, about 3300 Pa, about 3400 Pa, about 3500 Pa, about 3600 Pa, about 3700 Pa, about 3800 Pa, about 3900 Pa, about 4000 Pa, about 4100 Pa, about 4200 Pa, about 4300 Pa, about 4400 Pa, about 4500 Pa, about 4600 Pa, about 4700 Pa, about 4800 Pa, about 4900 Pa, about 5000 Pa, about 5100 Pa, about 5200 Pa, about 5300 Pa, about 5400 Pa, about 5500 Pa, about 5600 Pa, about 5700 Pa, about 5800 Pa, about 5900 Pa, about 6000 Pa, about 6100 Pa, about 6200 Pa, about 6300 Pa, about 6400 Pa, about 6500 Pa, about 6600 Pa, about 6700 Pa, about 6800 Pa, about 6900 Pa, about 7000 Pa, about 7100 Pa, about 7200 Pa, about 7300 Pa, about 7400 Pa, about 7500 Pa, about 7600 Pa, about 7700 Pa, about 7800 Pa, about 7900 Pa, about 8000 Pa, about 8100 Pa, about 8200 Pa, about 8300 Pa, about 8400 Pa, about 8500 Pa, about 8600 Pa, about 8700 Pa, about 8800 Pa, about 8900 Pa, about 9000 Pa, about 9100 Pa, about 9200 Pa, about 9300 Pa, about 9400 Pa, about 9500 Pa, about 9600 Pa, about 9700 Pa, about 9800 Pa, about 9900 Pa, or about 10,000 Pa or any elastic modulus in between a range defined by any two aforementioned values.

21. The crosslinked macromolecular matrix of any one of claims 1-20, wherein the matrix comprises a compression force value of about 10 gmf, about 20 gmf, about 30 gmf, about 40 gmf, about 50 gmf, about 60 gmf, about 70 gmf, about 80 gmf, about 90 gmf, about 100 gmf, about 110 gmf, about 120 gmf, about 130 gmf, about 140 gmf, about 150 gmf, about 160 gmf, about 170 gmf, about 180 gmf, about 190 gmf, about 200 gmf, about 210 gmf, about 220 gmf, about 230 gmf, about 240 gmf, about 250 gmf, about 260 gmf, about 270 gmf, about 280 gmf, about 290 gmf, about 300 gmf, about 310 gmf, about 320 gmf, about 330 gmf, about 340 gmf, about 350 gmf, about 360 gmf, about 370 gmf, about 380 gmf, about 390 gmf, about 400 gmf, about 410 gmf, about 420 gmf, about 430 gmf, about 440 gmf, about 450 gmf, about 460 gmf, about 470 gmf, about 480 gmf, about 490 gmf, about 500 gmf, about 510 gmf, about 520 gmf, about 530 gmf, about 540 gmf, about 550 gmf, about 560 gmf, about 570 gmf, about 580 gmf, about 590 gmf or about 600 gmf or any compression force value in between a range defined by any two aforementioned values.

22. The crosslinked macromolecular matrix of any one of claims 1-21, wherein the matrix comprises a compression force value of about 100 gmf, about 200 gmf, about 300 gmf, about 400 gmf, about 500 gmf or about 600 gmf or any compression force value in between a range defined by any two aforementioned values.

23. The crosslinked macromolecular mixture of any one of claims 1-22, wherein the hyaluronic acid is at a concentration of about 5 mg/ml, about 6 mg/ml, about 8 mg/ml, about 10 mg/ml, about 12 mg/ml, about 14 mg/ml, about 16 mg/ml, about 18 mg/ml, about 20 mg/ml, about 22 mg/ml, about 24 mg/ml, about 26 mg/ml, about 28 mg/ml, about 30 mg/ml, about 32 mg/ml, about 34 mg/ml or about 36 mg/ml or any concentration in between a range defined by any two aforementioned values.

24. The crosslinked macromolecular matrix of any one of claims 1-23, wherein the collagen comprises Type I collagen.

25. The crosslinked macromolecular matrix of any one of claims 1-24, wherein the collagen comprises Type II collagen.

26. The crosslinked macromolecular matrix of any one of claims 1-25, wherein the collagen comprises Type III collagen.

27. The crosslinked macromolecular matrix of any one of claims 1-26, wherein the collagen comprises 0% to 3% Type II collagen.

28. The crosslinked macromolecular matrix of any one of claims 1-27, wherein the collagen comprises 1%-3% Type I collagen.

29. The crosslinked macromolecular matrix of any one of claims 1-28, wherein the matrix comprises about 0% to about 3% type III collagen.

30. The crosslinked macromolecular matrix of any one of claims 1-29, wherein the collagen comprises about 97% to about 99% Type I collagen.

31. The crosslinked macromolecular matrix of any one of claims 1-30, wherein the collagen comprises a mixture of both Type I and Type III collagen.

32. The crosslinked macromolecular matrix of any one of claims 1-31, wherein the collagen comprises a concentration of about 1 mg/ml, about 2 mg/ml, about 4 mg/ml, about 6 mg/ml, about 8 mg/ml, about 10 mg/ml, about 12 mg/ml, about 14 mg/ml or any concentration in between a range defined by any two aforementioned values.

33. The crosslinked macromolecular matrix of any one of claims 1-32, wherein the crosslinked macromolecular matrix further comprising a salt.

34. The crosslinked macromolecular matrix of claim 33, wherein the crosslinked macromolecular matrix comprises NaCl in a range between about 50 mM to about 400 mM.

35. The crosslinked macromolecular matrix of any one of claims 1-34, wherein the crosslinked macromolecular matrix comprises NaCl, wherein the NaCl comprises a concentration of about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, or about 400 mM, or any concentration in between a range defined by any two aforementioned values.

36. The crosslinked macromolecular matrix of any one of claims 1-35, wherein the crosslinked macromolecular matrix comprises NaCl, wherein the NaCl comprises a concentration of about 150 mM.

37. The crosslinked macromolecular matrix of any one of claims 1-35, wherein the crosslinked macromolecular matrix comprises phosphate buffer of about 0.01M, NaCl of about 137 mM and KCl in a concentration of about 2.7 mM.

38. The crosslinked macromolecular matrix of any one of claims 1-37, wherein the crosslinked macromolecular matrix is formulated for injection or use with a needle and/or cannula.

39. The crosslinked macromolecular matrix of any one of claims 1-38, wherein the hyaluronic acid component has an average molecular weight of about 20,000 Daltons to about 10,000,000 Daltons.

40. The crosslinked macromolecular matrix of claim 39, wherein the hyaluronic acid component has an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

41. The crosslinked macromolecular matrix of any one of claims 1-40, wherein the hyaluronic acid comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

42. A composition comprising:
hyaluronic acid;
collagen;
lysine; and
a buffer;
wherein the composition is an aqueous hydrogel.

43. The composition of claim 42, wherein the hyaluronic acid is crosslinked to the collagen by at least one endogenous amine group on the collagen and/or by at least one amine group present on the lysine.

44. The composition of claim 42 or 43, wherein the composition further comprises lidocaine.

45. The composition of claim 44, wherein the lidocaine is at a concentration in between a range of about 0.15% (w/w) to about 0.45% (w/w) in the matrix.

46. The composition of claim 44 or 45, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.37% (w/w), about 0.39% (w/w), about 0.41% (w/w), about 0.43% (w/w), or about 0.45% (w/w) of the composition or any concentration in between a range defined by any two aforementioned values.

47. The composition of any one of claims 42-46, wherein the composition further comprises un-crosslinked HA.

48. The composition of any one of claim 47, wherein the un-crosslinked-crosslinked HA comprises a concentration of up to about 5% (w/w) within the composition.

49. The composition of any one of claim 47 or 48, wherein the un-crosslinked HA comprises a concentration of about 0% (w/w), about 1% (w/w), about 2% (w/w), about 3% (w/w), about 4% (w/w), about 5% (w/w) in the composition or any concentration in between a range defined by any two aforementioned values.

50. The composition of any one of claims 47-49, wherein the un-crosslinked HA comprises a concentration of about 1% (w/w) in the composition.

51. The composition of any one of claims 47-49, wherein the un-crosslinked HA comprises a concentration of about 2% (w/w) in the composition.

52. The composition of any one of claims 47-49, wherein the un-crosslinked HA comprises a concentration of about 5% (w/w) in the composition.

53. The composition of any one of claims 47-52, wherein the un-crosslinked HA, improves the extrudability of the composition.

54. The composition of any one of claims 42-53, wherein the buffer is phosphate buffered saline.

55. The composition of any one of claims 42-54, wherein the hyaluronic acid comprises an average molecular weight of about 20,000 Daltons to about 10,000,000 Daltons.

56. The composition of any one of claims 42-55, wherein the hyaluronic acid comprises a mixture of hyaluronic acid components with different molecular weights, wherein the mixture comprises hyaluronic acid with a molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

57. The composition of any one of claims 42-56, wherein the collagen comprises collagen type I.

58. The composition of any one of claims 42-57, wherein the collagen comprises collagen type II.

59. The composition of any one of claims 42-58, wherein the collagen comprises collagen type III.

60. The composition of any one of claims 42-59, wherein the composition comprises a viscosity of about 4,000 Pa S, about 4100 Pa S, about 4200 Pa S, about 4300 Pa S, about 4400 Pa S, about 4500 Pa S, about 4600 Pa S, about 4700 Pa S, about 4800 Pa S, about 4900 Pa S, about 5000 Pa S, about 5100 Pa S, about 5200 Pa S, about 5300 Pa S, about 5400 Pa S, about 5500 Pa S, about 5600 Pa S, about 5700 Pa S, about 5800 Pa S, about 5900 Pa S, about 6000 Pa S, about 6100 Pa S, about 6200 Pa S, about 6300 Pa S, about 6400 Pa S, about 6500 Pa S, about 6600 Pa S, about 6700 Pa S, about 6800 Pa S, about 6900 Pa S, about 7000 Pa S, about 7100 Pa S, about 7200 Pa S, about 7300 Pa S, about 7400 Pa S, about 7500 Pa S, about 7600 Pa S, about 7700 Pa S, about 7800 Pa S, about 7900 Pa S, about 8000 Pa S, about 8100 Pa S, about 8200 Pa S, about 8300 Pa S, about 8400 Pa S, about 8500 Pa S, about 8600 Pa S, about 8700 Pa S, about 8800 Pa S, about 8900 Pa S, about 9000 Pa S, about 9100 Pa, about 9200 Pa S, about 9300 Pa S, about 9400 Pa S, about 9500 Pa S, about 9600 Pa S, about 9700 Pa S, about 9800 Pa S, about 9900 Pa S, or about 10,000 Pa S or any viscosity in between a range defined by any two aforementioned values.

61. The composition of any one of claims 42-60, wherein the composition comprises a tan delta parameter (G″/G′) of about 0.01 to about 0.5.

62. The composition of any one of claims 42-61, wherein the composition comprises a tan delta parameter (G″/G′) of about 0.01, about 0.05, about 0.10, about 0.15, about 0.20, about 0.25, about 0.30, about 0.35, about 0.40, about 0.45 or about 0.50 or any tan delta parameter in between a range defined by any two aforementioned values.

63. The composition of any one of claims 42-62, wherein the composition is stable for about 6 months, about 12 months, about 18 months, about 24 months, about 30 months, or about 36 months, or any amount of time in between a range defined by any two aforementioned values.

64. The composition of any one of claims 42-63, wherein the composition is stable at about 4° C.

65. The composition of any one of claims 42-64, wherein the composition is stable at about 25° C.

66. The composition of any one of claims 42-65, wherein the composition has minimal degradation at about 6 months, about 12 months, about 18 months, about 24 months, about 30 months, or about 36 months, or any amount of time in between a range defined by any two aforementioned values.

67. A method of crosslinking hyaluronic acid and collagen comprising:
dissolving collagen, hyaluronic acid and lysine in an aqueous solution to form an aqueous pre-reaction solution, wherein the aqueous pre-reaction solution comprises a pH between about 4 and about 6; and
preparing a second solution comprising:
a water soluble carbodiimide; and
an N-hydroxysuccinimide or an N-hydroxysulfosuccinimide; and
adding the second solution to the aqueous pre-reaction solution to form a crosslinking reaction mixture; and
reacting the crosslinking reaction mixture by crosslinking the hyaluronic acid and the collagen with lysine;
wherein the hyaluronic acid is crosslinked to the collagen by at least one endogenous amine group on the collagen and/or by at least one amine group present on the lysine; and
wherein the HA and collagen undergo minimal degradation and the structure of the HA and collagen remains intact, thereby forming a crosslinked macromolecular matrix.

68. The method of claim 67, wherein the aqueous pre-reaction solution comprises a pH of about 4.0, about 4.5, about 5.0, about 5.5 or about 6.0, or any pH in between a range defined by any two aforementioned values.

69. The method of claim 67 or 68, wherein the method further comprises adding lidocaine to the crosslinked macromolecular matrix.

70. The method of claim 69, wherein the lidocaine is added to a concentration in between a range of about 0.15% (w/w) to about 0.45% (w/w) within the crosslinked macromolecular matrix.

71. The method of claim 69 or 70, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.37% (w/w), about 0.39% (w/w), about 0.41% (w/w), about 0.43% (w/w), or about 0.45% (w/w) of the matrix, or any concentration in between a range defined by any two aforementioned values.

72. The method of any one of claims 67-71, wherein the method further comprises providing an activating agent comprising a triazole, a fluorinated phenol, a succinimide, or a sulfosuccinimide.

73. The method of any one of claims 67-72, wherein the method is performed at a temperature of about 2° C., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., about 34° C., or about 36° C. or a temperature in between a range defined by any two aforementioned values.

74. The method of claim any one of claims 67-73, wherein the reacting step is performed between about 4 and about 35° C.

75. The method of any one of claims 67-74, wherein the reacting step is performed at about 4° C. or about 22° C.

76. The method of any one of claims 67-75, wherein the method further comprises purifying the crosslinked macromolecular matrix, wherein the purifying step is performed using dialysis.

77. The method of claim 76, wherein the purifying step is performed between 2° C.-30° C.

78. The method of claim 76 or 77, wherein the dialysis is performed at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., or any temperature in between a range defined by any two aforementioned values.

79. The method of any one of claims 76-78, wherein the purifying step is performed at about 2° C. to about 8° C.

80. The method of any one of claims 67-79, wherein the crosslinking reaction is performed at about 2° C. to about 35° C.

81. The method of any one of claims 67-80, wherein the crosslinking reaction is performed at about 2° C. to about 8° C.

82. The method of anyone of claims 67-81, wherein the method is performed below room temperature.

83. The method of any one of claims claim 67-82, wherein the pH of the crosslinking reaction mixture is between about 4.0 to about 6.0.

84. The method of any one of claims 67-83, wherein the pre-reaction solution comprises a salt, wherein the salt comprises sodium chloride at a concentration of about 50 mM, about 75 mM, about 100 mM, about 125 mM, about 150 mM, about 175 mM, about 200 mM, about 225 mM, about 250 mM, about 275 mM, about 300 mM, 325 mM, about 350 mM, about 375 mM, or about 400 mM, or any concentration in between a ranged defined by any two aforementioned values, in the crosslinking reaction mixture.

85. The method of any one of claims 67-84, wherein the water soluble carbodiimide is 1-ethyl-3-(3-dimethylaminopropyl)carbodiimide at a concentration of about 20 mM to about 200 mM in the crosslinking reaction mixture.

86. The method of claim 85, wherein the water soluble carbodiimide is 1-ethyl-3-(3-dimethylaminopropyl)carbodiimide at a concentration of about 20 mM, about 40 mM, about 60 mM, about 80 mM, about 100 mM, about 120 mM, about 140 mM, about 160 mM, about 180 mM or about 200 mM, or any concentration in between a range defined by any to aforementioned values.

87. The method of any one of claims 67-86, wherein the water soluble carbodiimide and hyaluronic acid is at a mole to mole ratio of water soluble carbodiimide: hyaluronic acid repeat unit between about 0.5 to about 2.0.

88. The method of claim 87, wherein the water soluble carbodiimide and hyaluronic acid is at a mole to mole ratio of water soluble carbodiimide: hyaluronic acid repeat unit of about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0.

89. The method of any one of claims 67-88, wherein the lysine and hyaluronic acid are at a mole:mole (lysine:HA repeat unit) ratio between about 0.01 to about 0.6.

90. The method of claim 89, wherein the lysine and hyaluronic acid are at a mole:mole (lysine:HA repeat unit) ratio of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59 or about 0.6.

91. The method of any one of claims 67-90, the method further comprising adding un-crosslinked HA to the crosslinked macromolecular matrix.

92. The method of claim 91, wherein the un-crosslinked HA is added to a concentration of up to 5% w/w within the crosslinked macromolecular matrix.

93. The method of claim 91 or 92, wherein the un-crosslinked HA is added to a concentration of about 0% (w/w), about 1% (w/w), about 2% (w/w), about 3% (w/w), about 4% (w/w), or about 5% (w/w) in the matrix, or any concentration in between a range defined by any two aforementioned values.

94. The method of any one of claims 91-93, wherein the un-crosslinked HA added to a concentration of about 1% (w/w) in the matrix.

95. The method of any one of claims 91-93, wherein the un-crosslinked HA is added to a concentration of about 3% (w/w) in the matrix.

96. The method of any one of claims 91-93, wherein the un-crosslinked HA is added to a concentration of about 5% (w/w) in the matrix.

97. The method of any one of claims 67-96, further comprising sterilizing the crosslinked macromolecular matrix, the method comprising:
transferring the crosslinked macromolecular matrix into a container, for steam sterilization; and
sterilizing the hydrogel by steam sterilization.

98. The method of claim 97, wherein the container is a syringe.

99. The method of any one of claims 67-98, wherein the method further comprises dialyzing the crosslinked macromolecular matrix, wherein the dialysis is through a membrane having a molecular weight cutoff of about 1000 Daltons to about 100,000 Daltons, and wherein the dialyzing is performed prior to sterilization.

100. The method of claim 99, wherein the dialysis is performed in phosphate buffered saline.

101. The method of any one of claims 67-100, wherein the hyaluronic acid in the pre-reaction solution hydrates for at least about 60 minutes prior to adding the second solution.

102. The method of any one of claims 67-101, wherein the crosslinking reaction mixture is performed for about 16 hours to about 24 hours.

103. A crosslinked macromolecular matrix prepared by a process of any one of claims 67-102.

104. A method of improving an aesthetic quality of an anatomic feature of a human being, the method comprising:
injecting a composition into a tissue of the human being to thereby improve the aesthetic quality of the anatomic feature;
wherein the composition comprises a crosslinked macromolecular matrix comprising:
hyaluronic acid;
lysine; and
collagen;
wherein the hyaluronic acid is crosslinked to the collagen by at least one endogenous amine group on the collagen and/or by at least one amine group present on the lysine.

105. The method of claim 104, wherein the crosslinked macromolecular matrix further comprises lidocaine.

106. The method of claim 104 or 105, wherein the crosslinked macromolecular matrix further comprises un-crosslinked HA.

107. The method of any one of claims 104-106, wherein the hyaluronic acid component has an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

108. The method of any one of claims 104-107, wherein the hyaluronic acid comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 1,000,000 Daltons and/or any hyaluronic acid with a molecular weight within a range in between any two aforementioned values.

109. The method of any one of claim 104-108, wherein the collagen comprises collagen type I and/or collagen type III.

110. A method of improving the appearance of an individual, the method comprising:
injecting a composition into a tissue of the individual at an injection site to thereby improve the aesthetic quality of an anatomic feature, wherein infiltrating cells from the tissue integrate into the composition within the injection site, depositing new collagen within the composition;
wherein the composition comprises a crosslinked macromolecular matrix comprising:
hyaluronic acid;
lysine; and
collagen;
wherein the hyaluronic acid is crosslinked to the collagen by at least one endogenous amine group on the collagen and/or by at least one amine group present on the lysine; and wherein the tissue injected by the composition is shown to have tissue integration and collagen deposition and blood vessel formation.

111. The method of claim 110, wherein the composition further comprises lidocaine.

112. The method of claim 110 or 111, wherein the composition further comprises un-crosslinked HA.

113. The method of any one of claims 110-112, wherein the composition is injected into a chin, jaw line, lips or nasolabial fold.

114. The method of claim 110-113, wherein the method improves symmetry among facial features.

115. The method of any one of claims 110-114, wherein the method enhances and restores volume to facial features.

116. The method of claim 115, wherein the method augments, corrects, restores or creates volume in the chin, jaw line, lips, or nasolabial fold.

117. The method of any one of claim 110-112, 114 or 115, wherein the composition is injected into tear troughs of the individual.

118. The method of any one of claims 110-117, wherein the composition is injected into an area comprising dermal atrophy and/or fat pad atrophy.

119. The method of any one of claims 110-118, wherein the method provides a natural look, feel and movement in the tissue receiving the injection, wherein the composition leads to increased infiltration of collagen from tissue surrounding the injection site.

120. The method of claim 119, wherein there is an enhanced duration of the composition as a result of tissue integration into the injection site.

121. The method of any one of claims 104-120, wherein the method improves hydration and elasticity of skin surrounding the injection site.

122. A method of increasing infiltration of collagen into a tissue, the method comprising:
injecting a composition into the tissue of an individual, thereby creating a dermal filler depot comprising the composition, wherein the composition comprises a crosslinked macromolecular matrix comprising:
hyaluronic acid; lysine; and collagen; wherein the hyaluronic acid is crosslinked to the collagen by at least one endogenous amine group on the collagen and/or by at least one amine group present on the lysine; and wherein cells from the tissue surrounding the dermal filler depot infiltrates the dermal filler depot comprising the composition, wherein the cells integrate into the composition and deposit new collagen into the composition, thereby creating infiltrated tissue within the composition and wherein blood vessels connect the infiltrated tissue within the composition to a blood supply of the individual's body.

123. The method of claim 122, wherein the matrix further includes lidocaine.

124. The method of claim 122 or 123, wherein the composition further comprises un-crosslinked HA.

125. The method of any one of claims 122-124, wherein the hyaluronic acid comprises an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any other molecular weight in between a range defined by any two aforementioned values.

126. The method of any one of claims 122-125, wherein the hyaluronic acid comprises a mixture of hyaluronic acid components with different molecular weights, wherein the mixture comprises hyaluronic acid with an average molecular weight of about 20,000 Daltons, about 40,000 Daltons, about 60,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,500,000 Daltons, about 2,000,000 Daltons, about 2,500,000 Daltons, about 3,000,000 Daltons, about 3,500,000 Daltons, about 4,000,000 Daltons, about 4,500,000 Daltons, about 5,000,000 Daltons, about 5,500,000 Daltons, about 6,000,000 Daltons, about 6,500,000 Daltons, about 7,500,000 Daltons, about 8,000,000 Daltons, about 8,500,000 Daltons, about 9,000,000 Daltons, about 9,500,000 Daltons and/or about 10,000,000 Daltons and/or any hyaluronic acid with a molecular weight within a range in between any two aforementioned values.

127. The method of any one of claims 122-126, wherein the collagen comprises collagen type I, collagen type II and/or collagen type III.

128. The method of any one of claims 122-127, wherein the composition comprises about 13 mg/ml hyaluronic acid.

129. The method of any one of claims 122-127, wherein the composition comprises about 20 mg/ml hyaluronic acid, about 22 mg/ml hyaluronic acid, about 24 mg/ml, about 26 mg/ml hyaluronic acid, about 28 mg/ml hyaluronic acid or about 30 mg/ml hyaluronic acid.
